(19) United States
(12) Patent Application Publication
(10) Pub. No.: US 2017/0119864 A1
(43) Pub. Date: May 4, 2017

(54) SACCHARIDE VACCINE FORMULATION

(71) Applicant: GLAXOSMITHKLINE BIOLOGICALS S.A., Rixensart (BE)

(72) Inventors: Erin WESTON, Laval (CA); Krikor TOROSSIAN, Laval (CA)

(21) Appl. No.: 14/910,710

(22) PCT Filed: Aug. 1, 2014
(86) PCT No.: PCT/EP2014/066591

§ 371 Date: Feb. 8, 2016

Related U.S. Application Data
(60) Provisional application No. 61/865,166, filed on Aug. 13, 2013.

(30) Foreign Application Priority Data
Aug. 8, 2013 (GB) 1314248.4

(51) Int. Cl. A61K 39/00

(52) U.S. Cl. CPC A61K 39/0011; A61K 2039/55561; A61K 2039/55555

(57) ABSTRACT

Substantially stable vaccine compositions are provided, as are methods for their use and manufacture.

### TECHNICAL FIELD

The present invention relates to improved formulations for saccharide vaccines comprising oppositely charged immunogenic molecules.

### BACKGROUND

MAG-Tn3 is a glyco-peptide antigen approximately 11 KDa in size. MAG-Tn3 is present is a substantial proportion of human cancers and is considered a candidate antigen for immunotherapy.

For immunotherapy, MAG-Tn3 could potentially be combined with an immunostimulant. One exemplary immunostimulant is a CpG oligodeoxynucleotide.

When manufacturing vaccines, a final liquid composition containing one or more of the immunogenic molecules is produced, commonly referred to as the ‘final bulk.’ For ease storage, the final bulk can be dried (for instance, by lyophilization). The dried vaccine, sometimes termed the lyophilization cake, may be reconstituted in a pharmaceutically acceptable solvent, such as water, buffer, etc., and may be termed the ‘final container.’

A MAG-Tn3/CpG final bulk could potentially be lyophilized to produce a final product for ease of storage. This final product could potentially be reconstituted in a buffer system or an adjuvant system for administration to the patient.

When formulating molecules for vaccine use, it is not certain that the composition will be stable. For instance, molecules may aggregate or precipitate under standard conditions. Even if such issues are overcome, physical or chemical degradation of one or more components may occur. Compositions and methods that overcome such limitations are needed. Further, molecules that are stable when formulated alone may undergo co-precipitation in the presence of other molecules.

### SUMMARY OF THE INVENTION

Substantially stable vaccine compositions are provided, the compositions comprising arginine; a counterion; a first immunogenic molecule comprising a Tn group, wherein the first immunogenic molecule has a net positive charge; and a second immunogenic molecule comprising an oligonucleotide, wherein the second immunogenic molecule has a net negative charge; the composition characterized in that when said composition comprises water (i) said first and second immunogenic molecule are substantially stable; and (ii) the pH of the resulting solution is less than 8.5. In certain aspects, the first immunogenic molecule is Mag-Tn3. In certain aspects, the second immunogenic molecule comprises a CpG oligonucleotide. In certain aspects, a portion of the arginine is present as the species of arginine monohydrochloride. In certain aspects, the composition is dried. In certain aspects, the composition comprises water.

In certain aspects, the composition further comprises an adjuvant composition comprising one or both the adjuvants MPL and QS21. In certain aspects, the adjuvant composition optionally further comprises liposomes.

Processes for making the substantially stable vaccine compositions are also provided, the processes comprising the steps of combining: arginine; a first immunogenic molecule comprising a Tn group, wherein the first immunogenic molecule has a net positive charge; and a second immunogenic molecule comprising an oligonucleotide, wherein the second immunogenic molecule has a net negative charge; wherein one or more of the preceding components are combined with a liquid comprising water and wherein the pH of said composition is 8.5 or less. Compositions produced by these processes are also provided.

Methods for treating a patient are provided, the methods comprising the steps of administering a composition as disclosed herein to a human. Uses are also provided, in particular the use of arginine monohydrochloride as an additive to a substantially stable vaccine composition.

Containers comprising the compositions as disclosed herein are also provided.

### BRIEF DESCRIPTION OF THE FIGURES

FIG. 1: Flow sheet of the separately mixed CpG7909 and MAG-Tn3 formulation procedure.

FIG. 2: Standard formulation flow sheet. Excipients used in the first step are listed in Table 2.

FIG. 3: Reduced SDS-PAGE of glutamic acid/lysine and glutamic acid/arginine and 1.0% w/v Empigen formulations after 24 hours at 4° C. MAG-Tn3 is the major band at approximately 15 KDa. The arrows highlight bands found in the pellet fractions, and the dashed and dotted circles highlight the increase in band intensity; the dashed lines being of higher intensity than the dotted. The control in MAG-Tn3 PB diluted in 10 mM Succinate pH 5.0 buffer, which was verified as the most stabilizing buffer system for MAG-Tn3 alone. This gel was stained using SilverExpress. NC: not centrifuged, SN: supernatant, P: re-suspended pellet fraction, MW: Molecular Weight Marker.

FIG. 4: SDS-PAGE of Tris-Maleate formulations after 24 hours at 4° C. MAG-Tn3 is the major band at approximately 15 KDa; the lower band corresponds to CpG at approximately 8 KDa. The arrows highlight bands found in the pellet fractions, and the dashed highlight the increase in band intensity. The control in MAG-Tn3 PB diluted in 10 mM Succinate pH 5.0 buffer, which was verified as the most stabilizing buffer system for MAG-Tn3 alone. This gel was stained using Silver Quest which stains CpG7909. NC: not centrifuged, SN: supernatant, P: re-suspended pellet fraction, MW: Molecular Weight Marker.

FIG. 5: Reduced SDS-PAGE of arginine and histidine formulation. MAG-tn3 is the major band at approximately 15 KDa; the lower band corresponds to CpG at approximately 8 KDa. The arrows highlight bands found in the pellet fractions. NC: not centrifuged, SN: supernatant, P: re-suspended pellet fraction.

FIG. 6: Reduced SDS-PAGE of L-arginine screening formulation. MAG-Tn3 is the major band at approximately 15 KDa; the lower band corresponds to CpG at approximately 8 KDa. NC: not centrifuged, SN: supernatant, P: re-suspended pellet fraction. A slightly more intense band (circled above) is observed in the pellet fraction of the 15 mM L-arginine formulation, than in the other formulations.

FIG. 7: HPLC-SEC Fluorescence chromatographic overlay of 15-35 mM L-arginine formulations.

FIG. 8: Reduced SDS-PAGE of L-arginine monohydrochloride screening formulation. MAG-Tn3 is the major band at approximately 15 KDa; the lower band corresponds to CpG at approximately 8 KDa. NC: not centrifuged, SN: supernatant, P: re-suspended pellet fraction. Slightly more intense bands in the pellet fractions are observed from 225 mM to 300 mM L-arginine monohydrochloride, circled above.

FIG. 9: HPLC-SEC Fluorescence chromatographic overlay of 100-200 mM L-arginine monohydrochloride formulations.

FIG. 10: Reduced SDS-PAGE of the dose range formulations. MAG-Tn3 is the major band at approximately 15 KDa; the lower band corresponds to CpG at approximately 8 KDa. The arrow indicates a slight band in the pellet fraction of the highest MAG-Tn3 concentration. NC: not centrifuged, SN: supernatant, P: re-suspended pellet fraction. MAG-Tn3 purified bulk control has been loaded in wells 2-4.

FIG. 11: Chromatographic overlay of the size exclusion profiles of 200-900 ug/mL MAG-Tn3 formulations.

FIG. 12: Reduced SDS-PAGE of the dose range formulations. MAG-Tn3 is the major band at approximately 15 KDa; the lower band corresponds to CpG at approximately 8 KDa. The arrow indicates a slight band in the pellet fractions of the higher MAG-Tn3 concentrations. The higher molecular weight bands present in well 18 are anomalous. NC: not centrifuged, SN: supernatant, P: re-suspended pellet fraction.

FIG. 13: HPLC-SEC Chromatogram overlay of 420, 270, and 180 ug CpG7909/dose mock final containers at T0.

FIG. 14: HPLC-SEC overlay of 420 ug CpG7909/dose MAG-Tn3 formulation at T0, 4 and 24 hour incubation at 25° C.

FIG. 15: HPLC-SEC Chromatogram of 180, 270, and 420 ug CpG/dose MAG-Tn3 formulations after a 24 hour 25° C. incubation.

### DETAILED DESCRIPTION

Applicants discovered that combining MAG-Tn3 and CpG molecules in solution causes an instantaneous co-precipitation and that when a lyophilized dry cake was re-constituted, it was not soluble when using standard excipients.

The inventors have surprisingly found that the inclusion of arginine, comprising a fraction of arginine monohydrochloride species, in a formulation for use with a first immunogenic molecule having net positive charge, in combination with a second immunogenic molecule having a net negative charge, allows for a suitable combination of stability and pH for use as an injectable vaccine in mammalian subjects.

### Compositions Comprising Arginine

In certain aspects herein the disclosure provides vaccine compositions comprising arginine, a counterion, a first immunogenic molecule having a net positive charge, and a second immunogenic molecule having a net negative charge, the composition is characterized in that when said composition comprises a pharmaceutically acceptable solvent (i) said first immunogenic molecule and said second immunogenic molecule are substantially stable; and (ii) the pH is less than 8.5.

In certain aspects, the first immunogenic molecule comprises a carbohydrate group. In certain aspects, the first immunogenic molecule comprises a Tn group. In certain aspects, the first immunogenic molecule comprises MAG-Tn3.

In certain aspects herein the second immunogenic molecule comprises an oligonucleotide. In certain aspects, the oligonucleotide is an immunostimulatory oligonucleotide. In certain aspects, the oligonucleotide is a CpG-containing oligonucleotide. In certain aspects, the oligonucleotide is CpG7909.

### Arginine

Arginine may be present as L- or D-forms, or a mixture of the two. L-Arginine is also known as L-(+)-Arginine2-amino-5-guanidinovaleric acid; 2-amino-5-guanidinovalerate; L-a-Amino-d-guanidinovalerateL-alpha-Amino-delta-guanidinovaleric acid; L-a-Amino-d-guanidinovaleric acid; N5-(aminoiminomethyl)-L-OrnithineL-alpha-Amino-delta-guanidinovalerate; 5-[(aminoiminomethyl)amino]-L-Norvaline(S)-2-Amino-5-[(aminoiminomethyl)amino]pentanoic acid; (S)-2-amino-5-[(aminoiminomethyl)amino]-Pentanoate(S)-2-Amino-5-[(aminoiminomethyl)amino]pentanoate; and (S)-2-amino-5-[(aminoiminomethyl)amino]-Pentanoic acid.

Arginine is represented by Formula I:

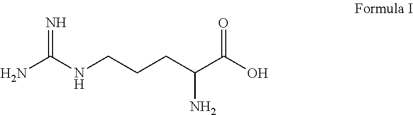 [SMILES: N=C(N)NCCCC(N)C(=O)O]

Arginine can be neutralized with hydrochloric acid or acids having a conjugate base other than chloride, resulting in Arginine•H—X, where X includes without limitation Cl⁻, SO₄⁻², and citrate.

In certain aspects herein, species of arginine include arginine monohydrochloride.

Arginine monohydrochloride may be present as L- or D-forms, or a mixture of the two. It is also known as, (2S)-2-Amino-5-[(aminoiminomethyl)amino]pentanoic acid monohydrochloride, arginine hydrochloride, and arginine•HCl. Arginine monohydrochloride may be manufactured by neutralizing arginine with hydrochloric acid. Arginine monohydrochloride is represented by Formula II.

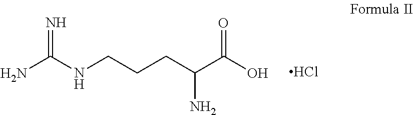 [SMILES: N=C(N)NCCCC(N)C(=O)O ; Cl]

Arginine and arginine•HCl are used in cell culture media and drug development. The amino acid arginine is used as a solution additive to stabilize proteins against protein-protein aggregation, especially in the process of protein refolding. See Baynes et al. (2005) Biochemistry 44:4919-4925; Tsumoto et al. (2004) Biotechnol. Prog. 20:1301-1308. As explained in Baynes, aggregation is the assembly of non-native protein conformations into multimeric states, often leading to phase separation and precipitation. The presence of arginine in solution was shown to slow protein-protein association reactions in two model systems: the association of insulin with a monoclonal antibody and the association of folding intermediates and aggregates of carbonic anhydrase II (CA). Arginine was used as a replacement for Human Serum Albumin to protect therapeutic proteins, including glycoproteins, from degradation. See Kim (2009) Biosci Biotechnol Biochem. 73:61-6.

Because arginine in solution is a polyprotic acid/base system, it is associated with four different protonation/charge states. A solution of arginine will comprise a mixture of species having different protonation states. These states are as follows:

1. “H₃B²⁺”,

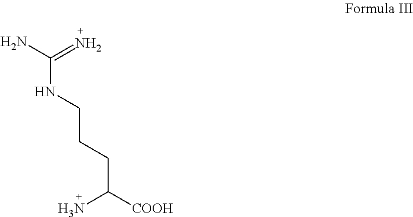 [SMILES: N=C(N)NCCCC(N)C(=O)O]

When the counter ion is 2 Cl⁻, the protonation state of Formula III is known as arginine dihydrochloride.

2. “H₂B⁺”,

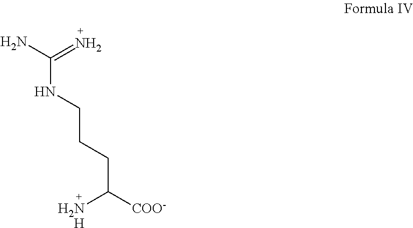

When the counter ion is Cl⁻, the protonation state of Formula IV is known as arginine monohydrochloride, or arginine•HCl.

3. “HB”

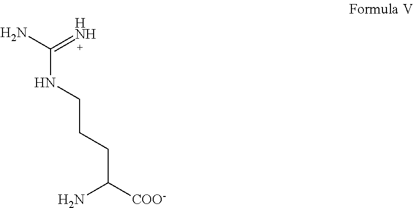

The protonation state of Formula V is known as arginine base.

4. “B⁻”

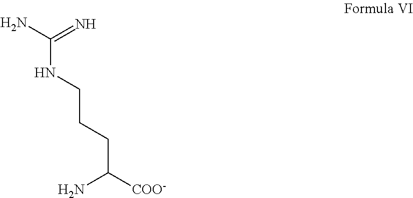

Where the counterion is Na+ or K+, the protonation state of Formula VI is known as sodium or potassium argininate.

Protonation/deprotonation of arginine proceeds according to the following scheme.

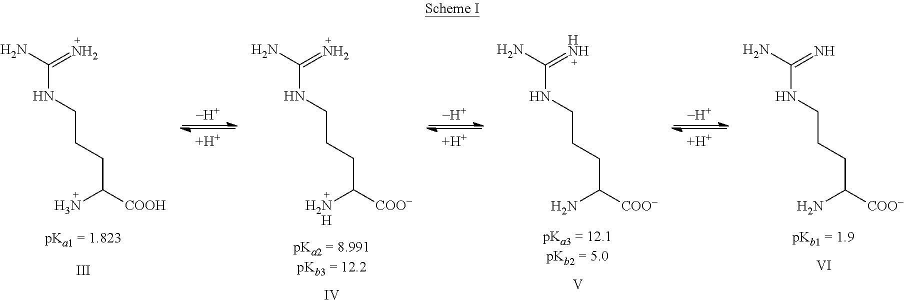

By “immunogenic molecule” is intended a molecule capable of inducing an immune response in a subject.

The term “molecules” herein includes without limitation macromolecules, oligomer molecules, and monomers. By “macromolecule” is intended polymeric molecules of high relative molecular mass, the structure of which essentially comprises the multiple repetition of units derived, actually or conceptually, from molecules of low relative molecular mass, including polysaccharides, polypeptides, nucleic acids, and the like, as well as non-polymeric molecules with large molecular mass such as lipids and macrocycles. By “oligomer molecule” is intended a molecule of intermediate relative molecular mass, the structure of which essentially comprises a small plurality of units derived, actually or conceptually, from molecules of lower relative molecular mass. By “monomer” is intended a molecule which can undergo polymerization.

By “net charge” of a molecule is intended the arithmetic sum of positive and negative charges on the molecule at a given pH or pH range. A molecule having a “net positive charge” will have a majority of positive charges at a given pH or pH range; likewise, a molecule with a “net negative charge” will have a majority of negative charges at a given pH or pH range. The applicable pH or pH range is the pH or pH range of the solution comprising the relevant molecule.

### Carbohydrate Groups Comprising a Tn Group

In certain aspects herein the disclosure provides immunogenic molecules comprising carbohydrate groups or carbohydrate antigens.

By a “carbohydrate group” is intended a carbohydrate portion of a molecule chemically linked to another portion of the molecule. Thus, a carbohydrate group may be attached to another carbohydrate molecule or to another category of molecule, such as a protein (or peptide). Exemplary molecules having carbohydrate groups include oligosaccharides, polysaccharides, glycopeptides, glycoproteins, and the like, of which some may be carbohydrate antigens.

By “carbohydrate antigen” is intended a saccharide-based antigen, including bacterial capsular polysaccharides, tumor-associated carbohydrate antigens, and the like.

In certain aspects herein the disclosure provides immunogenic molecules comprising a Tn group.

By “Tn” or “Tn goup” is intended a member of the glycophorin family as described in Morrelli (2011) Eur. J. Org. Chem. 5723-5777. Tn may be described as an N-Acetylgalactosamine linked to either a serine or threonine residue via a glycosidic bond. Thus, a molecule comprising Tn will have one or more Tn groups.

In certain aspects herein the disclosure provides immunogenic molecules comprising MAG-Tn3.

“MAG-Tn3” is disclosed in EP2500033A1 and has the structure following structure:

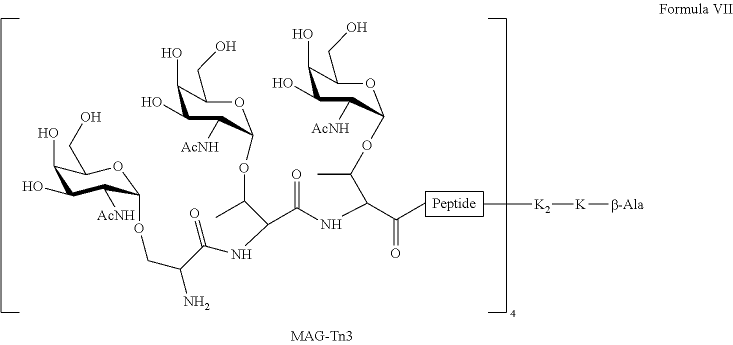

Thus, MAG-Tn3 corresponds to a carbohydrate peptide conjugate B4-T4-M of Formula IV:

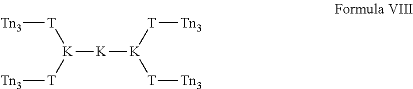

### Wherein

KKK is the dendritic polyLysine core (M),
T is the peptidic CD4+ T cell epitope having the following sequence: QYIKANSKFIGITEL
Tn3 is the tri-Tn B cell epitope having the following sequence: (α-GalNAc)Ser-(α-GalNAc)Thr-(α-GalNAc)Thr.
MAG-Tn3 has an estimated pI of 9.8-10, and is therefore very positively charged at neutral pH.

### Oligonucleotides

By “immunostimulatory oligonucleotide” is intended an oligonucleotide that comprises an immunostimulatory DNA motif. Immunostimulatory DNA motifs are described in Sato et al. (1996) Science 273:352. Exemplary immunostimulatory oligonucleotide include CpG-containing oligonucleotides (in which the CpG dinucleotide is unmethylated), which induce a predominantly Th1 response. Such oligonucleotides are well known and are described, for example, in WO 96/02555, WO 99/33488 and U.S. Pat. Nos. 6,008,200 and 5,856,462.

Exemplary CpG-containing oligonucleotides include the following specific sequences:

| CpG | | SEQ |
|---|---|---|
| No. | Sequence | ID NO |
| 1826 | TCC ATG ACG TTC CTG ACG TT | 1 |
| 1758 | TCT CCC AGC GTG CGC CAT | 2 |
| 1212 | ACC GAT GAC GTC GCC GGT GAC GGC | 3 |
| | ACC ACG | |
| 2006/ | TCG TCG TTT TGT CGT TTT GTC GTT | 4 |
| 7909 | | |
| 1668 | TCC ATG ACG TTC CTG ATG CT | 5 |
| 5456 | TCG ACG TTT TCG GCG CGC GCC G | 6 |

CpG7909 is a synthetic single stranded 24-mer oligodeoxynucleotide with a phophorothioate backbone of approximately 8 KDa and has 23 negative charges at neutral pH.

The term “substantially stable” is intended to describe a solution wherein the solute does not precipitate out of solution. That is, once a finite period of time has passed after the solute of interest has been dissolved in the solution, T1, more than 70% of the solute will remain in solution, i.e. more than 71, 72, 73, 74, 75, 76, 77, 78, 79, 80, 81, 82, 83, 84, 85, 86, 87, 88, 89, 90, 91, 92, 93, 94, 95, 96, 97, 98, 99% or more of the solute will remain in solution.

The finite period of time, T1, may be any period of time longer than 1 hour, i.e. 1, 2, 3, 5, 10, 20, 20, 30, 50, 75, 100, 150, 200, 250, 500, 750, 1000, 1500 or more hours.

Stability may be assessed by numerous methods including, for instance, by measuring loss upon filtration, determining the presence of the solute of interest in a pellet fraction (by SDS-PAGE or equivalent method), or by determining the aggregation profile for the solute of interest.

In certain aspects herein, the composition is characterized in that when the composition comprises water the pH of the composition is within a range wherein the upper limit pH is less than 8.5; less than 8.4; less than 8.3; less than 8.2; less than 8.1; less than 8.0; less than 7.9; less than 7.8; less than 7.7; less than 7.6; or less than 7.5 and the lower limit is greater than 7.4; greater than 7.5; greater than 7.6; greater than 7.7; greater than 7.8; greater than 7.9; greater than 8.0; greater than 8.1; greater than 8.2; greater than 8.3; or greater than 8.4. In certain aspects herein, the pH is between 7.4 and 8.5, inclusive; between 7.5 and 8.5, inclusive; between 7.6 and 8.3, inclusive; between 7.7 and 8.3, inclusive; between 7.8 and 8.3, inclusive; between 7.9 and 8.3, inclusive; between 8.0 and 8.3, inclusive, between 8.1 and 8.3, inclusive.

Methods for achieving the desired pH of the composition include neutralizing the arginine solution with a suitable acid, such as hydrochloric acid, or combining a requisite amount of arginine and arginine•H—X to produce the desired pH in solution. In certain aspects, the arginine•H—X is arginine monohydrochloride. The ratio of arginine:arginine monohydrochloride necessary to yield a desired pH can be readily calculated using known methods, such as the Henderson-Hasselbalch equation, wherein

pH=pKα+log([deprotonated Arg]/[protonated Arg]) Equation 1.

For instance, Chart 1 sets forth the calculated pH resulting from various molar ratios of arginine:arginine-HCl.

CHART 1
Calculated pH values using equation 1 for solutions comprising various
molar ratios of Arg:ArgHCl (Arg pKa = 8.991).

| | | Arginine concentration held | | | | Arginine monohydrochloride | | | |
|---|---|---|---|---|---|---|---|---|---|
| | Molar | constant | | | | concentration held constant | | | |
| | Ratio of | | [Arginine- | | [Arginine- | | [Arginine- | | [Arginine- |
| | Arg:ArgHCl | [Arginine] | HCl] | [Arginine] | HCl] | [Arginine] | HCl] | [Arginine] | HCl] |
| pH | (mM:mM) | (mM) | (mM) | (mM) | (mM) | (mM) | (mM) | (mM) | (mM) |
| 7.5 | 0.032 | 15 | 465 | 40 | 1239 | 7 | 220 | 3 | 100 |
| 7.6 | 0.041 | 15 | 369 | 40 | 984 | 9 | 220 | 4 | 100 |
| 7.7 | 0.051 | 15 | 293 | 40 | 782 | 11 | 220 | 5 | 100 |
| 7.8 | 0.064 | 15 | 233 | 40 | 621 | 14 | 220 | 6 | 100 |
| 7.9 | 0.081 | 15 | 185 | 40 | 493 | 18 | 220 | 8 | 100 |
| 8.0 | 0.102 | 15 | 147 | 40 | 392 | 22 | 220 | 10 | 100 |
| 8.1 | 0.129 | 15 | 117 | 40 | 311 | 28 | 220 | 13 | 100 |
| 8.2 | 0.162 | 15 | 93 | 40 | 247 | 36 | 220 | 16 | 100 |
| 8.3 | 0.204 | 15 | 74 | 40 | 196 | 45 | 220 | 20 | 100 |
| 8.4 | 0.256 | 15 | 58 | 40 | 156 | 56 | 220 | 26 | 100 |
| 8.5 | 0.323 | 15 | 46 | 40 | 124 | 71 | 220 | 32 | 100 |

As will be appreciated, the actual pH of the solutions produced may be confirmed and adjusted by routine means, such as a pH meter. In certain aspects herein, the actual pH is no more than ±0.2 pH units from the calculated pH value or is no more than ±0.2 pH units outside of the calculated pH range.

In certain aspects herein, the composition is characterized in that when said composition comprises water, the arginine comprises the following species:

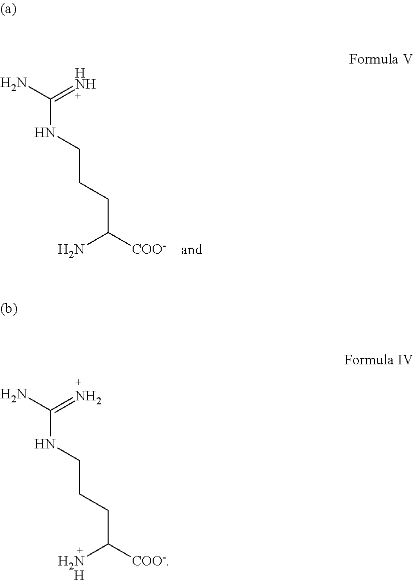

In certain aspects, the molar ratio of species (a) to species (b) is between 7:220 (0.032) and 71:220 (0.323). In certain aspects, the molar ratio of species (a) to species (b) is between 1:11 (0.091) and 1:5 (0.200). In certain aspects, Formula V is at least 14 mM, and the molar ratio of the species of (a) Formula V to the species of (b) Formula IV is within a range selected from the group consisting of: (a) between 0.091 and 0.200; (b) between 0.032 and 0.323; (c) between 0.041 and 0.323; (d) between 0.051 and 0.256; (e) between 0.064 and 0.256; and (f) between 0.081 and 0.204.

### Cryoprotectants

In certain aspects herein the composition comprises a cryoprotectant.

By “cryoprotectant” is intended a substance used to protect biomolecules from freezing conditions, such as those encountered during freeze drying or lyophilization. Exemplary cryoprotectants include carbohydrates, such as the saccharide sucrose, sugar alcohols such as mannitol, surface active agents such as the polysorbates, as well as glycerol and dimethylsulfoxide. Exemplary carbohydrates include saccharides and disaccharides. Exemplary disaccharides include sucrose and trehalose.

### Adjuvants

In certain aspects herein, the compositions and methods also include an adjuvant composition comprising one or more adjuvants. In the context of an immunogenic composition suitable for administration to a subject, such as a human subject, for the purpose of eliciting an immune response, the adjuvant composition is selected to elicit a Th1 biased immune response. The adjuvant composition is typically selected to enhance a Th1 biased immune response in the subject, or population of subjects, to whom the composition is administered.

A “Th1” type immune response is characterized by the induction of CD4+ T helper cells that produce IL-2 and IFN-γ. In contrast, a “Th2” type immune response is characterized by the induction of CD4+ helper cells that produce IL-4, IL-5, and IL-13.

### TLR4 Modulators

One suitable adjuvant is a TLR4-modulator. One example is a non-toxic derivative of lipid A, is monophosphoryl lipid A or more particularly 3-Deacylated monophoshoryl lipid A (3D-MPL). 3D-MPL is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

In other embodiments the lipopolysaccharide can be a β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. Nonetheless, each of these references is incorporated herein by reference. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments the adjuvant is a synthetic derivative of lipid A, some of which are described as TLR-4 agonists, and include, but are not limited to: OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026); OM 294 DP (3S,9R)-3-[(R)-dodecanoyloxytetradecanoylamino]-4-oxo-5-aza-9(R)-[(R)-3-hydroxytetradecanoylamino]decan-1,10-diol, 1,10-bis(dihydrogenophosphate) (WO 99/64301 and WO 00/0462); and OM 197 MP-Ac DP (3S-,9R)-3-[(R)-dodecanoyloxytetradecanoylamino]-4-oxo-5-aza-9-[(R)-3-hydroxytetradecanoylamino] decan-1,10-diol, 1-dihydrogenophosphate 10-(6-aminohexanoate) (WO 01/46127).

### Saponin Adjuvants

Other adjuvants that can be used in immunogenic compositions herein, e.g., on their own or in combination with 3D-MPL, or another adjuvant described herein, are saponins, such as QS21.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1, which are incorporated herein by reference. Other saponins which have been used in systemic vaccination studies include those derived from other plant species such as Gypsophila and Saponaria (Bomford et al., Vaccine, 10(9):572-577, 1992).

Such formulations comprising QS21 and cholesterol have been shown to be successful Th1 stimulating adjuvants when formulated together with an antigen.

### Other Molecules

In certain aspects, one or more other molecules may be included in the compositions herein, including nonionic surfactants and emulsifiers, such as polysorbate 80 (Tween™ 80, available from ICI Americas, Inc.), excipients, buffers, and the like, such as sodium phosphate, potassium phosphate, etc.

### Liquids

In certain aspects, the compositions herein comprise water. In certain aspects, the arginine is present at a concentration of at least 15 mM, i.e. 16, 17, 18, 19, 20, 21, 22, 23, 24, 25, 30, 35, 40 mM, or higher. In certain aspects, the arginine monohydrochloride is present at a concentration of at least 45 mM, i.e., 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mM, or higher.

### Final Bulk

In certain aspects, the composition comprises water and the first immunogenic molecule comprises MAG-Tn3 and is present at a concentration of less than or equal to 900 μg/ml. In certain aspects, the composition comprises water and the first immunogenic molecule comprises MAG-Tn3 and is present at a concentration of between 60-900 μg/mL inclusive. In certain aspects, the composition comprises water and the oligonucleotide is present at a concentration of less than 1140 μg/ml. In certain aspects, the composition comprises water and the oligonucleotide is present at a concentration of 760-1140 μg/ml, inclusive. In certain aspects, the composition comprises water and the oligonucleotide is present at a concentration of 950 μg/ml. In certain aspects, the composition comprises water and the arginine is present at a concentration of 25 mM. In certain aspects, the composition comprises water and arginine monohydrochloride is present at a concentration of 187.5 mM. In certain aspects, the composition comprises water and (i) between 60-900 μg/mL MAG-Tn3, inclusive; (ii) 950 μg/mL CpG 7909 (SEQ ID NO:4); (iii) 25 mM arginine; (iv) 187.5 mM arginine monohydrochloride; (v) 0.108 w/v Polysorbate 80; and (vi) 5% w/v sucrose.

### Dried Cake

In certain aspects, the composition is dried, and the ratio of arginine:arginine monohydrochloride is 20:150 (mol:mol) or 1.74:15.8 (wt:wt). In certain aspects, the dried composition comprises between 380-570 μg CpG 7909 (SEQ ID NO:4). In certain aspects, the dried composition comprises (i) between 30-450 μg MAG-Tn3, inclusive; (ii) 475 μg CpG 7909 (SEQ ID NO:4); (iii) 0.87 mg arginine; (iv) 7.9 mg arginine monohydrochloride; (v) 0.216 mg Polysorbate 80; and (vi) 10 mg sucrose.

### Final Container

In certain aspects, the composition comprises water and the first immunogenic molecule comprises MAG-Tn3 and is present at a concentration of less than 720 μg/ml. In certain aspects, the composition comprises water and the first immunogenic molecule comprises MAG-Tn3 and is present at a concentration of between 48-720 μg/mL inclusive. In certain aspects, the composition comprises water and the oligonucleotide is present at a concentration of 608-912 μg/ml, inclusive. In certain aspects, the composition comprises water and the oligonucleotide is present at a concentration of 760 μg/ml. In certain aspects, the composition comprises water and the arginine is present at a concentration of 20 mM. In certain aspects, the composition comprises water and arginine monohydrochloride is present at a concentration of 150 mM. In certain aspects, the composition comprises water and (i) between 48-720 μg/mL MAG-Tn3, inclusive; (ii) 760 μg/mL CpG 7909 (SEQ ID NO:4); (iii) 20 mM arginine; (iv) 150 mM arginine monohydrochloride; (v) 0.0864% w/v Polysorbate 80; and (vi) 4% w/v sucrose. In certain aspects, the composition comprises water and (i) between 48-720 μg/mL MAG-Tn3, inclusive; (ii) 760 μg/mL CpG 7909 (SEQ ID NO:4); (iii) 20 mM arginine; (iv) 150 mM arginine monohydrochloride; (v) 0.0864% w/v Polysorbate 80; and (vi) 4% w/v sucrose; (vii) 150 mM NaCl; (viii) 8 mM KH₂PO₄ and 2 mM Na₂HPO₄; (ix) 50 μL/mL MPL; (x) 100 μg/mL liposomes; and (xi) 100 μg/mL QS21.

### Processes and Methods

In certain aspects, processes for making the substantially stable vaccine compositions herein are provided, comprising combining components thereof in a single step, or in several steps. In certain aspects, processes for making the substantially stable vaccine compositions herein are provided, comprising a step of combining components comprising arginine; a first immunogenic molecule comprising a Tn group, wherein the first immunogenic molecule has a net positive charge; and a second immunogenic molecule comprising an oligonucleotide, wherein the second immunogenic molecule has a net negative charge. In certain aspects, processes for making the substantially stable vaccine compositions herein are provided, comprising the steps of combining components comprising arginine; a first immunogenic molecule comprising a Tn group, wherein the first immunogenic molecule has a net positive charge; and a second immunogenic molecule comprising an oligonucleotide, wherein the second immunogenic molecule has a net negative charge. In certain aspects, arginine may comprise a species having a counterion. In certain aspects, processes for making the substantially stable vaccine compositions herein are provided, comprising a step of combining components comprising arginine; arginine monohydrochloride; a first immunogenic molecule comprising a Tn group, wherein the first immunogenic molecule has a net positive charge; and a second immunogenic molecule comprising an oligonucleotide, wherein the second immunogenic molecule has a net negative charge. In certain aspects, processes for making the substantially stable vaccine compositions herein are provided, comprising the steps of combining components comprising arginine; arginine monohydrochloride; a first immunogenic molecule comprising a Tn group, wherein the first immunogenic molecule has a net positive charge; and a second immunogenic molecule comprising an oligonucleotide, wherein the second immunogenic molecule has a net negative charge. In certain aspects, processes for making the substantially stable vaccine compositions herein are provided, comprising the steps of combining the components of arginine; arginine monohydrochloride; a first immunogenic molecule comprising a Tn group, wherein the first immunogenic molecule has a net positive charge; and a second immunogenic molecule comprising an oligonucleotide, wherein the second immunogenic molecule has a net negative charge. One or more of these components are combined with a liquid comprising water. Standard methods can be utilized for combining these components. Typically, a stock solution comprising the arginine and arginine monohydrochloride species is prepared, which is then combined with stock solutions of the other components. Alternatively, arginine monohydrochloride may be prepared by neutralizing arginine with hydrochloric acid. Where a different counterion is desired, similar approaches using a different acid may be used.

For instance, when the immunogenic molecules are MAG-Tn3 and CpG, respectively, the following formulation protocol can be followed: Using stock solutions of 500 mM L-Arginine and 1M L-Arginine mono-hydrochloride, formulations can made by adding 31.3 mM L-Arginine and 187.5 mM L-Arginine monohydrochloride to a 5% Sucrose solution in water for injection (available from Thermo-Fisher). MAG-Tn3 and CpG are both commercially available and the manufacturer's protocol for preparing stock solutions of these molecules may be followed. CpG7909 (available from Agilent) is then added to the solution at a concentration of 1050 μg/mL. The solution is then magnetically stirred for 5 minutes at 150 rpm. MAG-Tn3 obtained from Lonza Braine is then added to the solutions at concentrations ranging from 250-1125 μg/mL. The solutions are then stirred magnetically for another 5 minutes at 150 rpm. The formulations are then diluted 1.25 times in a solution of 50 mM Na₂HPO₄/KH₂PO₄ 150 mM NaCl pH 6.1. See Examples 3 and 4.

In certain aspects, processes for drying the composition are provided. Standard techniques can be used to dry the composition, including freeze drying, lyophilization, and the like. Standard lyophilization protocols may be used. In one aspect, a 64 hour lyophilization cycle is used, wherein a product temperature of below −34.5° C. is avoided during the primary drying phase of the freeze cycle. For instance, conditions may include an initial freezing of 1 hour at −52° C., followed by primary drying where the temperature is increased to between −27° C. and −37° C. in 2.5-3.5 hours. The temperature can then be held for approximately 27 to 37 hours. The primary drying temperature can then be ramped up to between −23° C. and −33° C. over a period of 4.25-5.75 hours. This temperature can be held for 4.25-5.75 hours. All of primary drying may be performed with a chamber pressure of 45 μbar (34 mTorr). Secondary drying may begin with the temperature being ramped up to between 32-42° C. in 5.4-6.6 hours at a chamber pressure of 15-75 μbar (11-56 mTorr) and held for 10.8-13.2 hours at a pressure of 10-45 μbar (8-34 mTorr).

In certain aspects, processes are provided for combining the compositions with a liquid comprising water, wherein the liquid further comprises an adjuvant composition comprising one or more adjuvants, wherein at least one of the adjuvants is selected from the group consisting of MPL and QS21. In certain aspects, processes for reconstituting the dried compositions are provided, comprising the steps of combining the dried composition with a liquid comprising water, wherein the liquid further comprises an adjuvant composition comprising one or more adjuvants selected from the group consisting of MPL and QS21. In certain aspects, the adjuvant further comprises liposomes.

In certain aspects, products according to the foregoing processes are provided.

In certain aspects, the compositions herein may be present in one or more containers. For instance, a first container may comprise arginine and the first and second immunogenic molecules, while a second container may comprise an adjuvant composition comprising one or more adjuvants selected from the group consisting of MPL and QS21. Alternatively, one container may comprise arginine, the first and second immunogenic molecules, and an adjuvant composition comprising one or more adjuvants selected from the group consisting of MPL and QS21. In certain aspects, kits comprising one or more containers comprising the compositions herein are provided.

In certain aspects, methods for treating a patient comprising the steps of administering a composition described herein are provided. In certain aspects, methods of inducing an immunogenic response comprising the steps of administering a composition to a human are provided. Administration may be by injection.

In some aspects are provided the use of arginine monohydrochloride as an additive to stabilize a vaccine composition.

In some aspects, the compositions provided herein are for use in medicine, such as for use in inducing an immune response. In some aspects, the compositions are for use in the treatment of cancer, wherein the first immunogenic molecule is Mag-Tn3. In some aspects the compositions are for use in the treatment of breast cancer, wherein the first immunogenic molecule is Mag-Tn3.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. Thus, where a pH is indicated to be at least pH 7.5, it is intended that the pH be understood to be at least approximately (or “about” or “˜”) pH 7.5, i.e., at least 7.5±0.2 pH units.

The invention will be further described by reference to the following, non-limiting, figures and examples.

### EXAMPLES

### Example 1. Excipient Screening: Determination of a Suitable Buffer System for Mag-Tn3 and CpG7909

The targeted dose for MAG-Tn3 antigen was set at 500 μg/dose. In the presence of the immunostimulant CpG7909, the MAG-Tn3 antigen co-precipitates instantaneously. A multitude of buffer systems were tried in an effort to solubilize this antigen-immunostimulant combination. This report will detail the numerous buffer and excipient combinations tried in an attempt to solubilize MAG-Tn3 and CpG7909. MAG-Tn3 is a glyco-peptide antigen approximately 11 KDa in size. It has an estimated pI of 9.8-10, and is therefore very positively charged at neutral pH. This antigen is to be combined with the immunostimulant CpG7909 to be formulated as a lyophilized vaccine which would be reconstituted in the adjuvant system known as AS01B. CpG7909 is a synthetic single stranded 24-mer oligodeoxynucleotide with a phophorothioate backbone of approximately 8 KDa and has 23 negative charges at neutral pH. The combination of these two molecules without the addition of excipients causes an instantaneous co-precipitation.

Initial attempts at solubilizing MAG-Tn3 and CpG7909 were performed at the final bulk level, where the compatibility with the AS01B buffer system was not assessed. These experiments indicated that with the addition of histidine at a concentration of 18.75 mM in the final bulk resulted in a soluble formulation. It was discovered when this lyophilized final product was reconstituted in the adjuvant buffer system that the formulation was no longer soluble and the problem of co-precipitation had not been entirely resolved.

The objective of the experiments described in this report was to find a way in which both MAG-Tn3 and CpG7909 could be formulated together in a soluble vaccine formulation when reconstituted in AS01B buffer. The previous experiments had demonstrated the need to assess the formulation stability in the adjuvant buffer from the start and the experiments described in this report were performed as such.

### Experimental Procedure.

Preparation of Formulations:

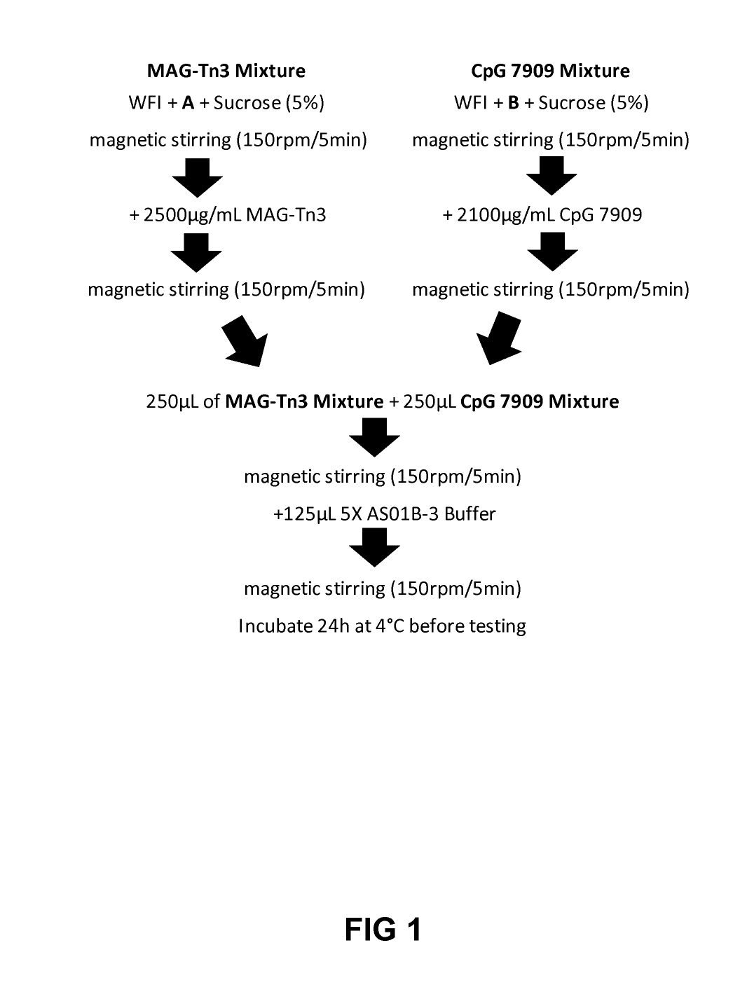

Two types of formulations were performed. The first type of formulation, illustrated in FIG. 1. MAG-Tn3 and CpG7909 are formulated separately and then combined. Excipient A is added to a 5% sucrose solution in water for injection (Thermo-Fisher) at varying concentrations as indicated in Table 1, and then magnetically stirred for 5 minutes at 150 rpm. MAG-Tn3 obtained from Lonza Braine is then added to the solution at a concentration of 2500 μg/mL for a final dose of 500 μg. This solution is magnetically stirred for 5 minutes at 150 rpm. In a separate container excipient B is added to a 5% sucrose solution in water for injection and magnetically stirred for 5 minutes at 150 rpm. CpG7909 (Agilent) is then added to this solution at a concentration of 2100 μg/mL. The solution is then stirred magnetically for 5 minutes at 150 rpm. The two solutions, MAG-Tn3 Mixture and CpG Mixture are then combined at a 1:1 ratio and then magnetically stirred for 5 minutes at 150 rpm. This formulation is then diluted 1.25 times in a solution of 50 mM Na₂HPO₄/KH₂PO₄ 150 mM NaCl pH 6.1 (AS01B Buffer). The formulations are then incubated for twenty-four hours at 4° C. before being analyzed.

TABLE 1
Buffer compositions tested using the separate mixing method outlined in
FIG. 1.

| Formulation Objective | A | B |
|---|---|---|
| Glutamic Acid 7 mM + 20 mM Arginine | Glutamic Acid 17.5 mM | 50 mM Arginine |
| Glutamic Acid 7 mM + 20 mM Histidine | Glutamic Acid 17.5 mM | 50 mM Histidine |
| Sodium Octanoate 1.3% w/v + 20 mM | Sodium Octanoate | 50 mM Arginine |
| Arginine | 3.25% w/v | |
| Sodium Octanoate 1.3% w/v + 20 mM | Sodium Octanoate | 50 mM Histidine |
| Histidine | 3.25% w/v | |
| 0.25, 1.0% w/v Empigen | 0.625, 2.5% w/v | 0.625, 2.5% w/v |
| | Empigen | Empigen |
| 0.25, 1.0% w/v SB3-13 | 0.625, 2.5% w/v SB3-14 | 0.625, 2.5% w/v |
| | | SB3-15 |
| 10 mM Glutamic Acid- 10 mM Histidine | 25 mM Glutamic Acid | 25 mM Histidine |
| 50 mM Glutamic Acid- 10 mM Histidine | 125 mM Glutamic Acid | 25 mM Histidine |
| 50 mM Glutamic Acid- 50 mM Histidine | 125 mM Glutamic Acid | 125 mM Histidine |
| 10 mM Glutamic Acid- 10 mM Lysine | 25 mM Glutamic Acid | 25 mM Lysine |
| 10 mM Glutamic Acid- 50 mM Lysine | 25 mM Glutamic Acid | 125 mM Lysine |
| 50 mM Glutamic Acid- 10 mM Lysine | 125 mM Glutamic Acid | 25 mM Lysine |
| 50 mM Glutamic Acid- 50 mM Lysine | 125 mM Glutamic Acid | 125 mM Lysine |
| 10 mM Glutamic Acid - 10 mM Arginine | 25 mM Glutamic Acid | 25 mM Arginine |
| 10 mM Glutamic Acid - 50 mM Arginine | 25 mM Glutamic Acid | 125 mM Arginine |
| 50 mM Glutamic Acid - 10 mM Arginine | 125 mM Glutamic Acid | 25 mM Arginine |
| 50 mM Glutamic Acid - 50 mM Arginine | 125 mM Glutamic Acid | 125 mM Arginine |
| Ammonium Acetate 100 mM | Ammonium Acetate | Ammonium |
| | 100 mM | Acetate 100 mM |
| 10, 50 mM Tris-Maleate | 10, 50 mM Tris-Maleate | 10, 50 mM Tris-Maleate |
| 20, 30, 40, 50, 75 mM Tris 10 mM | 25 mM Maleate | 50, 75, 100, 125, |
| Maleate | | 187.5 mM Tris |
| 10 mM Tris-50 mM Maleate | 125 mM Maleate | 25 mM Tris |
| 50 mM Tris-20, 30, 40 mM Maleate | 50, 75, 100 mM Maleate | 125 mM Tris |

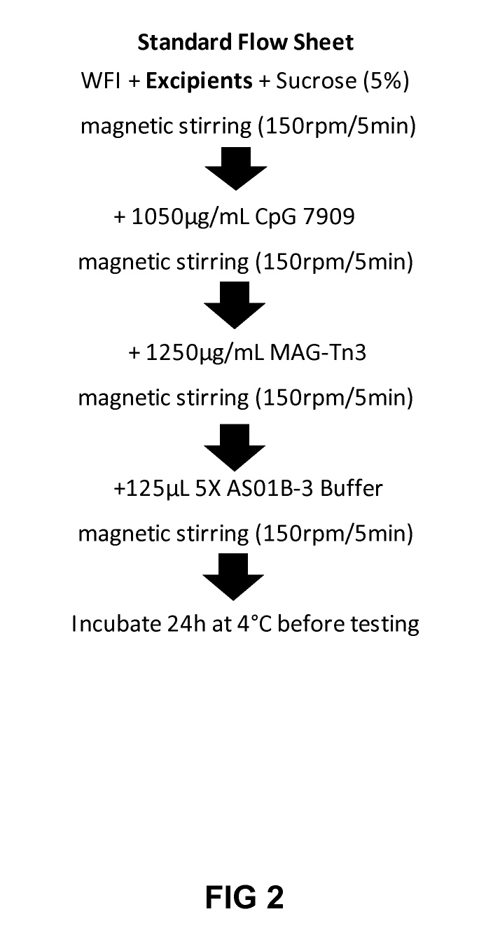

The second method used for formulating was a standard flow sheet (FIG. 2). All excipients, see Table 2 for list, are added to a 5% sucrose solution in water for injection at varying concentrations. CpG7909 is then added to the solution at a concentration of 1050 μg/mL. The solution was then magnetically stirred for 5 minutes at 150 rpm. MAG-Tn3 is then added to the solutions at a concentration of 1250 μg/mL for a final dose of 500 μg. The solutions were then stirred magnetically for another 5 minutes at 150 rpm. The formulations were then diluted 1.25 times in a solution of 50 mM Na₂HPO₄/KH₂PO₄ 150 mM NaCl pH 6.1. All formulations were then incubated for twenty-four hours at 4° C. before being analyzed.

TABLE 2
List of excipients formulated in the standard flow sheet. The
concentrations indicated are the concentrations found in the final
reconstituted container.
Excipients
15 mM Histidine + 40 mM Tris + 0.01%(w/v), 0.1%(w/v) Tween
15 mM Histidine + 40 mM Tris + 0.01% (w/v), 0.1% (w/v) Lutrol
15 mM Histidine + 40 mM Tris + 0.01% (w/v), 0.1% (w/v) Sodium
Octanoate
50 mM Tris-Maleate
80 mM Histidine
α-cyclodextrin 0.25 (w/v), 2.5%(w/v)
Benzalkonium Chloride 0.025(w/v), 0.125%(w/v)

All samples were first visually inspected. If any precipitation or opaqueness of solution was noticed no other testing was performed. If the formulation was translucent additional analyses were performed such as BCA, HPLC-SEC, and SDS-PAGE. BCA was performed to assess MAG-Tn3 content in the formulations. The standard Pierce protocol and reagents were used. Size-exclusion chromatography was performed using a Waters 2996 HPLC equipped with a fluorescence detector (Waters) to assess the aggregation profiles of the resulting formulations using a TSKgel G3000PWxl column (Tosoh Bioscience LLC) and a mobile phase of 200 mM NaCl (EMD). SDS-PAGE analysis was done using 4-12% Bis-Tris gels (Invitrogen) and MES running buffer (Invitrogen). The gels were stained using Invitrogen's SilverExpress or SilverQuest. Centrifuged and non-centrifuged samples were run on gel to assess the presence of precipitate in the formulations. Centrifugation was performed for 15 minutes at 18 000 g. The supernatant was extracted after which the pellet was re-suspended in 1×LDS sample buffer (Invitrogen) and both fractions were run on gels. pH (Orion), and visual inspection analyses were also performed.

Most of the formulations were not successful and the excipient used could be ruled out on the basis of visual inspection, often without beginning the 24 hour incubation. The results of the visual aspect analysis can be seen in Table 3.

TABLE 3
Summary table of visual aspect and turbidity results. Samples that
precipitated were not tested further.

| Formulation Flow | | Visual | |
|---|---|---|---|
| sheet | Excipient and concentrations | Aspect | pH |
| Standard | 15 mM Histidine + 40 mM Tris + 0.01%, 0.1% | PPT | — |
| | Tween | | |
| Standard | 15 mM Histidine + 40 mM Tris + 0.01%, 0.1% | PPT | — |
| | Lutrol | | |
| Standard | 15 mM Histidine + 40 mM Tris + 0.01%, 0.1% | PPT | — |
| | Sodium Octanoate | | |
| Standard | 50 mM Tris-Maleate | PPT | — |
| Standard | 80 mM Histidine | PPT | — |
| Standard | α-cyclodextrin 0.25, 2.5% w/v | PPT | — |
| Standard | Benzalkonium Chloride 0.025, 0.125% w/v | PPT | — |
| Separate mixing | Glutamic Acid 7 mM + 20 mM Arginine | Translucent | 9.08 |
| Separate mixing | Glutamic Acid 7 mM + 20 mM Histidine | PPT | — |
| Separate mixing | Sodium Octanoate 1.3% w/v + 20 mM Arginine | PPT | — |
| Separate mixing | Sodium Octanoate 1.3% w/v + 20 mM Histidine | PPT | — |
| Separate mixing | 0.25% w/v Empigen | PPT | — |
| Separate mixing | 1.0% w/v Empigen | Translucent | 5.69 |
| Separate mixing | 0.25, 1.0% w/v SB3-13 | PPT | — |
| Separate mixing | 10 mM Glutamic Acid- 10 mM Histidine | PPT | — |
| Separate mixing | 50 mM Glutamic Acid- 10 mM Histidine | PPT | — |
| Separate mixing | 50 mM Glutamic Acid- 50 mM Histidine | PPT | — |
| Separate mixing | 10 mM Glutamic Acid- 10 mM Lysine | Translucent | 8.04 |
| Separate mixing | 10 mM Glutamic Acid- 50 mM Lysine | Translucent | 9.56 |
| Separate mixing | 50 mM Glutamic Acid- 10 mM Lysine | PPT | — |
| Separate mixing | 50 mM Glutamic Acid- 50 mM Lysine | Translucent | 9.32 |
| Separate mixing | 10 mM Glutamic Acid - 10 mM Arginine | Translucent | 8.16 |
| Separate mixing | 10 mM Glutamic Acid - 50 mM Arginine | Translucent | 9.57 |
| Separate mixing | 50 mM Glutamic Acid - 10 mM Arginine | PPT | — |
| Separate mixing | 50 mM Glutamic Acid - 50 mM Arginine | Translucent | 9.25 |
| Separate mixing | Ammonium Acetate 100 mM | PPT | — |
| Separate mixing | 10, 50 mM Tris-Maleate | PPT | — |
| Separate mixing | 20, 30, 40 mM Tris 10 mM Maleate | PPT | — |
| Separate mixing | 50, 75 mM Tris 10 mM Maleate | Translucent | 8.16, |
| | | | 8.53 |
| Separate mixing | 10 mM Tris-50 mM Maleate | PPT | — |
| Separate mixing | 50 mM Tris-20, 30, 40 mM Maleate | PPT | — |

PPT: Precipitation.

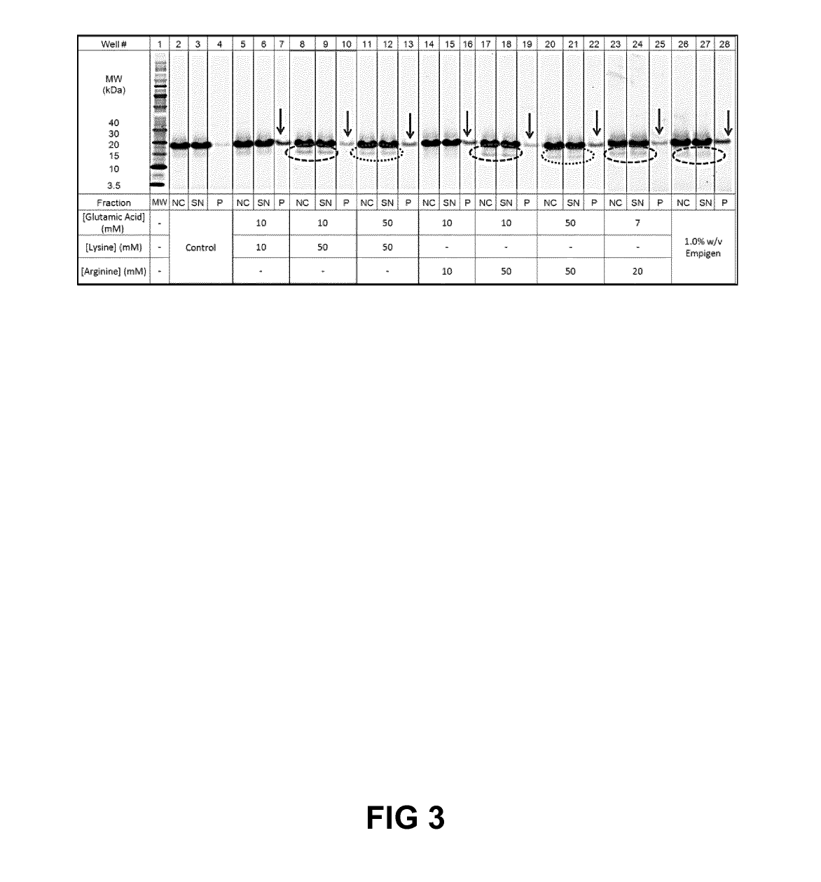

For those formulations that did not precipitate, analyses were performed after the 24 hour incubation at 4° C. The SDS-PAGE results indicated that though precipitation was not observed upon visual inspection, most of the formulations had slightly precipitated highlighted in FIG. 3 by arrows. Though a band is present in the pellet fraction, it does not appear to be enough material to see a corresponding decrease in band intensity in the supernatant fractions.

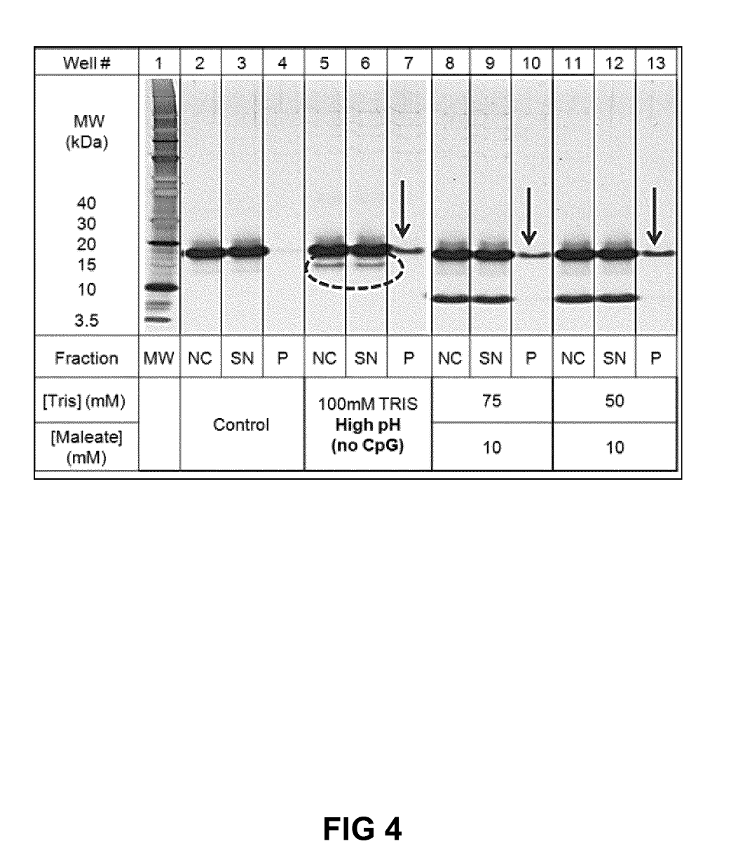

Circled in either a dotted or dashed line is potentially of more concern, as this highlights an increase in intensity of the degradation band which is only slightly present in the control. 10 mM glutamic acid with either 10 mM lysine or 10 mM arginine appears to have a similar band intensity of the degradation band as the control. This may be indirectly due to pH, as both of these formulations have a pH of approximately 8, whereas the other non-surfactant formulations have very high pH of greater than 9. This is confirmed further where the two tris-maleate formulations barely exhibit a degradation band, whereas the high pH tris formulation has quite an intense and well defined degradation band. See FIG. 4.

The results from the BCA assay to determine MAG-Tn3 content were not promising, see Table 4. It should be noted that the BCA assay was not optimized for each buffer system used. In spite of this, useful data can be extracted. It is interesting to note that the two formulations that have the lowest loss of MAG-Tn3 content, with the exception of the Empigen formulation, are the 10 mM glutamic acid with either 50 mM arginine or 50 mM lysine. These two formulations have pHs of approximately 9.5 and intense degradation bands by SDS-PAGE. The tris-maleate formulations though somewhat promising by SDS-PAGE are not by BCA. Losses of approximately 50% are observed for these two formulations.

TABLE 4
MAG-Tn3 antigen content determined by BCA and HPLC-SEC results. The
percent loss is based on a theoretical MAG-Tn3 concentration of 826 μg/mL. The
MAG-Tn3 concentration aimed for in these formulations was 500 μg/dose or
1000 μg/mL based on the mass of the purified bulk used and the reconstitution
volume. It was later discovered that the percent glyco-peptide content should be
used in determining the concentration in order to account for the water and counter-
ion content. For this lot of MAG-Tn3 the glyco-peptide content was 82.6%.

| | MAG-Tn3 Concentration | | |
|---|---|---|---|
| | by BCA (μg/mL) | | % Monomer |
| Excipient and concentrations | Total | % Loss | by HPLC-SEC |
| Glutamic Acid 7 mM + 20 mM Arginine | 542.4 | 34.3 | 100 |
| 1.0% w/v Empigen | 957.9 | 0.0 | 88.6 |
| 10 mM Glutamic Acid- 10 mM Lysine | 507.3 | 38.6 | 100 |
| 10 mM Glutamic Acid- 50 mM Lysine | 746.2 | 9.7 | 100 |
| 50 mM Glutamic Acid- 50 mM Lysine | 551.9 | 33.2 | 100 |
| 10 mM Glutamic Acid - 10 mM Arginine | 454.7 | 45.0 | 100 |
| 10 mM Glutamic Acid - 50 mM Arginine | 808.7 | 2.1 | 100 |
| 50 mM Glutamic Acid - 50 mM Arginine | 537.7 | 34.9 | 100 |
| 50 mM Tris 10 mM Maleate | 406.8 | 50.8 | — |
| 75 mM Tris 10 mM Maleate | 433.5 | 47.5 | — |

The Empigen formulation though low in pH and exhibiting decent recoveries, has a large high molecular weight aggregate in its size-exclusion chromatogram (data not shown). This aggregate is represents approximately 11% by peak area.

Many formulations were tried in an effort to solubilize MAG-Tn3 and CpG7909, yet none were completely successful. MAG-Tn3 appears to be most soluble at high pH, however pHs of greater than 8.5 are known to cleave sugars. Since MAG-Tn3 is a glycol-peptide with the Tn sugar being the antigenic portion, high pHs need to be avoided. At higher pH there is an increase in the degradation band by SDS-PAGE, though the identity of this band has yet to be determined it may be a deglycosylated MAG-Tn3 or MAG.

It is interesting to note that when equal concentrations of anionic and cationic buffers are used they exhibit the same range of MAG-Tn3 loss, 38.6 and 33.2% for 10 and 50 mM glutamic acid and lysine formulations. The protein loss is a lot less, 9.7%, when an excess of cationic buffer, in this case lysine, is used. A similar trend is seen for the glutamic acid-arginine combinations. A possible reason for the formulation instabilities may be the presence of competing ions. If glutamic acid is interacting with arginine or lysine, it would then reduce the amount of arginine or lysine free in solution to interact and stabilize CpG and prevent the co-precipitation with MAG-Tn3. Future experiments will explore this possibility.

### Example 2. Histidine Versus Arginine: Determination of a Buffer System at Lower Dose Target

The targeted dose for MAG-Tn3 antigen was set at 500 μg/dose. In the presence of the immunostimulant CpG7909, the MAG-Tn3 antigen co-precipitates instantaneously. A multitude of buffer systems were tried in an effort to solubilize this antigen-immunostimulant combination however none was found to be adequate. Histidine and Arginine were however the most promising buffers of the systems explored. In this report the experiments carried out to determine which buffer system would perform better at a lower dose will be described. The essential results obtained from this experiment were that the antigen-immunostimulant combination could be formulated as a soluble solution however the targeted dose of antigen would need to be reconsidered.

The MAG-Tn3 antigen was to be formulated at 500 μg/dose in the presence of the immunostimulant CpG7909 at a concentration of 840 μg/mL as a co-lyophilization. A buffer compatibility study was performed in a previous experiment, in which it was clearly demonstrated that the MAG-Tn3 was incompatible with CpG7909 as the solution precipitated upon addition of the MAG-Tn3 to the CpG solution. MAG-Tn3 has a theoretical pI between 9.8 and 10; hence it is therefore very positively charged at lower pH. Additionally, CpG has 23 negative charges at lower pH. Therefore, when the 2 components are combined, a co-precipitation occurs. Many buffer systems were tried in an attempt to resolve this, arginine and histidine seemed to provide some help against precipitation, but it was not complete. The objective of this paper is to describe the experiments performed with histidine and arginine to help solubilize the MAG-Tn3 antigen in the presence of the immunostimulant CpG7909.

In the experiment in this report various concentrations of arginine and histidine were screened with the MAG-Tn3 dose set at its mid dose amount of 100 μg and the stability of these samples were then monitored.

### Experimental Procedure

Preparation of Formulations:

Arginine (Sigma-Aldrich) or histidine (Sigma-Aldrich) was added to a 5% sucrose solution in water for injection (Thermo-Fisher) at concentrations of 15.2, 31.3, 62.5, and 125 mM. CpG7909 (Agilent) was then added to the solution at a concentration of 1050 μg/mL. The solution was then magnetically stirred for 5 minutes at 150 rpm. MAG-Tn3 obtained from Lonza Braine was then added to the solutions at a concentration of 250 μg/mL for a final dose of 100 μg. The solutions were then stirred magnetically for another 5 minutes at 150 rpm. The formulations were then diluted 1.25 times in a solution of 50 mM Na₂HPO₄/KH₂PO₄ 150 mM NaCl pH 6.1. All Formulations were then incubated for twenty-four hours at 4° C. before being analyzed.

Analyses:

RP-HPLC was performed to assess MAG-Tn3 content in the formulations before and after filtration with a 0.2 μm syringe filter. A Waters 2996 HPLC equipped with UV detection was used with a Poros R 1/10 column from Applied Biosciences and a 0-100% acetonitrile in 0.1% triflouroacetic acid gradient. Size-exclusion chromatography was performed using a Waters 2996 HPLC equipped with a fluorescence detector (Waters) to assess the aggregation profiles of the resulting formulations using a TSKgel G3000PWxl column (Tosoh Bioscience LLC) and a mobile phase of 200 mM NaCl. SDS-PAGE analysis was done using 4-12% Bis-Tris gels (Invitrogen) and MES running buffer (Invitrogen). The gels were stained using Invitrogen's SilverQuest. Centrifuged and non-centrifuged samples were run on gel to assess the presence of precipitate in the formulations. Centrifugation was performed for 15 minutes at 18 000 g. The supernatant was extracted after which the pellet was re-suspended in 1×LDS sample buffer (Invitrogen) and both fractions were run on gels. pH (Orion), and visual inspection analyses were also performed.

All formulations were translucent and particle-free upon storage at 4° C. However after the 24 hour incubation, the histidine solutions were all found to be slightly turbid by visual inspection. The pH readings, Table 5, did not provide any information with regards to product stability; however, it should be noted that the histidine formulations have a narrower pH range than the arginine formulations with the same concentrations.

TABLE 5
pH of formulations. pH Readings were taken for all of the solutions.

| | | pH | |
|---|---|---|---|
| | | Arginine | Histidine |
| 12.5 | mM | 8.4 | 6.7 |
| 25 | mM | 9.4 | 6.9 |
| 50 | mM | 9.8 | 7.1 |
| 100 | mM | 10.1 | 7.3 |

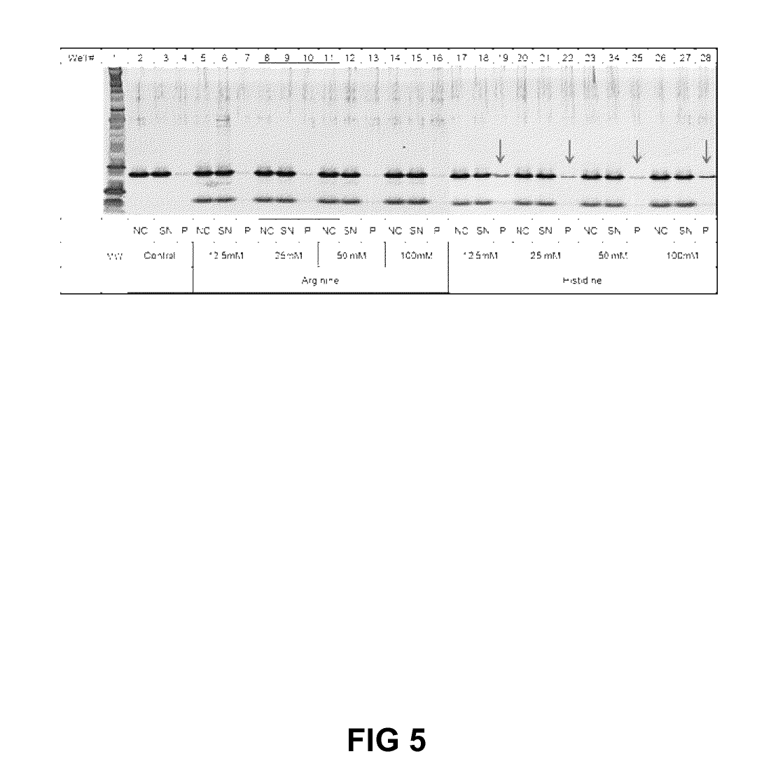

The SDS-PAGE results, seen in FIG. 5, confirm the results observed visually. The histidine formulations all contain slight bands in the pellet fractions, whereas the Arginine formulations do not.

The HPLC-SEC results did not provide any additional data. All formulations were found to be monomeric and no significant shifts in retention time were observed; thus, suggesting that the MAG-Tn3 which remained soluble was in monomeric form.

TABLE 6
MAG-Tn3 antigen content. MAG-Tn3 antigen content was
determined by RP-HPLC before and after filtration with a 0.2 μm
syringe filter to assess insoluble aggregate content.

| | MAG-Tn3 Concentration by RP-HPLC (μg/mL) | | | | | |
|---|---|---|---|---|---|---|
| | Arginine | | | Histidine | | |
| | | After | % | | After | % Re- |
| | Total | Filtration | Recovery | Total | Filtration | covery |
| 12.5 mM | 202.9 | 188.1 | 92.7 | 209.3 | 138.4 | 66.1 |
| 25 mM | 209.1 | 183.2 | 87.6 | 197.6 | 130.7 | 66.2 |
| 50 mM | 207.9 | 184.1 | 88.5 | 198.4 | 131.5 | 66.3 |
| 100 mM | 204.9 | 197.4 | 96.3 | 202.1 | 131.9 | 65.3 |

The RP-HPLC results, seen in Table 6, confirmed the results obtained from the SDS-PAGE. The histidine formulations all exhibit an approximate content loss of 33% after filtration, as a result of insoluble aggregates. Due to the significant loss of MAG-Tn3 content with the histidine formulations, arginine was selected as the buffer system of choice.

### Results

Both histidine and arginine buffer systems help solubilize the antigen MAG-Tn3 in the presence of the immunostimulant CpG7909. The cationic nature of these buffer systems helps stabilize the anionic CpG, preventing it from co-precipitating with the MAG-Tn3 antigen. The lower pKa of histidine would make it a more favorable buffer as it would result in formulations with a lower pH; however, it is not as effective as arginine at solubilizing the two major components which was demonstrated by the 33% loss in antigen content when assayed by RP-HPLC.

The side chain of arginine has a pKa of 12.48 making the pH of the final solutions quite high, between pH 8 and 10. The long term stability of MAG-Tn3 in this buffer system may be hindered, as it is a glyco-peptide and high pH favors de-glycosylation. An additional buffer component will need to be added to the formulation in order to lower the pH to a range that will be more favorable with regards to the antigen's stability.

The choice of an additional buffer component will be difficult as seen in Example 1. It would appear that the presence of an anionic buffer adds a competing ion for the cationic buffer system, in this case arginine, thereby liberating CpG so that it can in turn co-precipitate with MAG-Tn3. An ideal candidate for an anionic buffer would be one that can lower the pH to below 8.5, while not competing with arginine or an anionic buffer that would have more affinity for MAG-Tn3 than CpG has for the antigen.

### Example 3. L-Arginine L-Arginine Monohydrochloride Concentrations in the MAG-Tn3 Vaccine Formulation

The glyco-peptide MAG-Tn3 can be formulated in a soluble vaccine with the immunostimulant CpG7909 at a dose of 300 μg/mL in an L-arginine buffer system. Previous experiments have shown that L-arginine concentrations between 12.5 and 100 mM were sufficient to solubilize the vaccine formulation. This report will detail the experiments performed to determine the exact L-arginine and L-arginine monohydrochloride concentrations used in the MAG-Tn3 vaccine formulation.

The MAG-Tn3 vaccine formulation was initially targeted for a dose of 500 μg in a 500 μL injection volume containing 420 μg of the immunostimulant CpG7909. This formulation was not stable and resulted in a co-precipitation of the immunostimulant and the antigen. The vaccine formulation was made soluble with the use of L-arginine in combination with lowering the targeted dose to 300 μg of MAG-Tn3. It had been shown in previous experiments that L-arginine monohydrochloride was effective at lowering the formulation pH to 8.5 all the while maintaining a soluble formulation. The need to keep the pH below 8.5 is due to the potential deglycoslylation of the Tn sugar group from the molecule. The Tn sugar is the antigenic portion of the molecule, its loss would be have a significant impact on immunogenicity.

The objective of this report is to describe the experiments performed in order to optimize the L-arginine and L-arginine monohydrochloride concentrations for maximum vaccine stability. L-arginine concentrations of 12.5 to 40 mM were tested with L-arginine monohydrochloride concentrations varying in order to maintain a pH around 8.5. Once the L-arginine concentration was established, the L-arginine monohydrochloride concentrations were screened to determine the optimal pH to ensure maximum stability of the vaccine formulation. In both cases the lowest concentration that provides the best stability will be selected as the concentration of choice.

### Experimental Procedures

Preparation of Formulations:

Using stock solutions of 500 mM L-Arginine (EMD) and 1M L-Arginine monohydrochloride (Sigma Aldrich), formulations were made by adding 15.6-40 mM L-Arginine and 60-140 mM L-Arginine monohydrochloride to a 5% Sucrose (EMD) solution in water for injection (Thermo-Fisher) for the first part of the experiment in which the L-arginine was determined. In the following experiment, 25 mM L-Arginine and 125-375 mM L-Arginine mono-hydrochloride was added to a 5% Sucrose solution in water for injection. CpG7909 (Agilent) was then added to the solution at a concentration of 1050 μg/mL. The solution was then magnetically stirred for 5 minutes at 150 rpm. MAG-Tn3 obtained from Lonza Braine was then added to the solutions at a concentration of 750 μg/mL. The solutions were then stirred magnetically for another 5 minutes at 150 rpm. The formulations were then diluted 1.25 times in a solution of 50 mM Na₂HPO₄/KH₂PO₄ 150 mM NaCl pH 6.1. All Formulations were then incubated for twenty-four hours at 4° C. before being analyzed. Chemicals were provided from Sigma-Aldrich.

Analyses:

RP-HPLC was performed to assess MAG-Tn3 content in the formulations before and after filtration with a 0.2 μm syringe filter. A Waters 2996 HPLC equipped with UV detection was used with a Poros R 1/10 column from Applied Biosciences and a 0-100% acetonitrile in 0.1% triflouroacetic acid gradient. Size-exclusion chromatography was performed using a Waters 2996 HPLC equipped with a fluorescence detector (Waters) to assess the aggregation profiles of the resulting formulations using a TSKgel G3000PWxl column (Tosoh Bioscience LLC) and a mobile phase of 200 mM NaCl. SDS-PAGE analysis was done using 4-12% Bis-Tris gels (Invitrogen) and MES running buffer (Invitrogen). The gels were stained using Invitrogen's SilverQuest. Centrifuged and non-centrifuged samples were run on gel to assess the presence of precipitate in the formulations. Centrifugation was performed for 15 minutes at 18 000 g. The supernatant was extracted after which the pellet was re-suspended in 1×LDS sample buffer (Invitrogen) and both fractions were run on gels. Turbidity (HACH), pH (Orion), and visual inspection analyses were also performed.

All formulations were found to be translucent and particle-free after the 24 hour incubation at 4° C. by visual analysis.

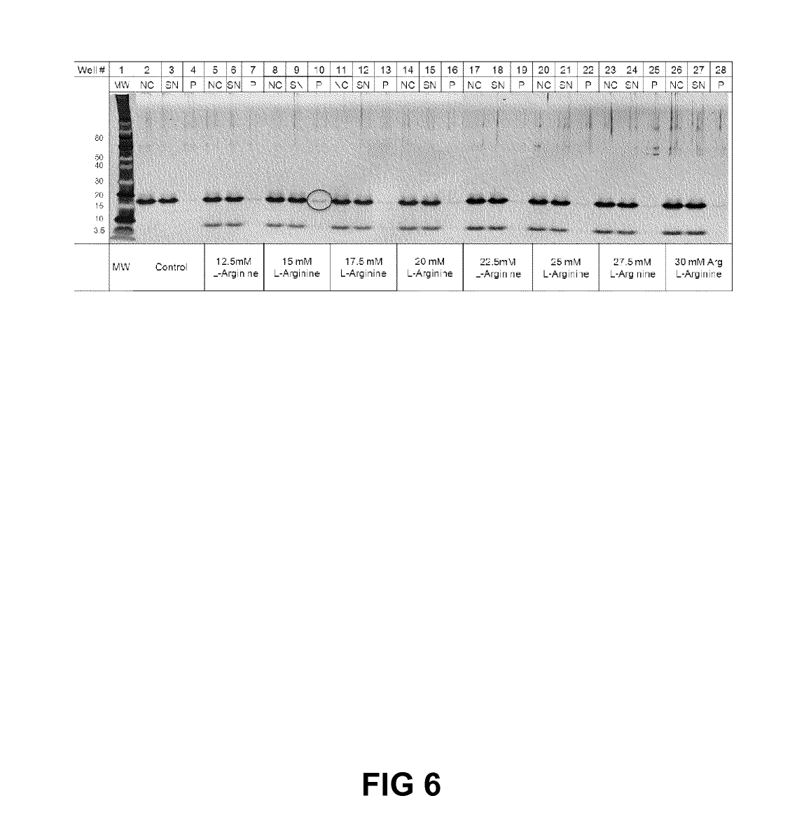

The SDS-PAGE results, visualized in FIG. 6, indicate that all formulations from 12.5 to 30 mM L-arginine are stable, with the exception of the 15 mM 1-arginine formulation which has a slightly more intense band in its pellet fraction.

The turbidity and pH results are similar between formulations and no significant difference was noted, as seen below in Table 7.

TABLE 7
L-arginine screening. A summary of results from
L-arginine screening is provided.

| | | | | | | % |
|---|---|---|---|---|---|---|
| L-Arginine | | | MAG-Tn3 Concentration | | | Monomer |
| Con- | | | by RP-HPLC (μg/mL) | | | by |
| centration | Turbidity | | | After | % | HPLC- |
| (mM) | (NTU) | pH | Total | Filtration | Recovery | SEC |
| 12.5 | 0.465 | 8.0 | 736.9 | 583.6 | 79.2 | 94.4 |
| 15.0 | 0.836 | 8.2 | 871.0 | 782.8 | 89.9 | 94.0 |
| 17.5 | 0.442 | 8.2 | 782.6 | 804.4 | 102.8 | 94.3 |
| 20.0 | 0.417 | 8.3 | 755.3 | 792.0 | 104.9 | 94.4 |
| 22.5 | 0.468 | 8.4 | 824.2 | 826.1 | 100.2 | 94.7 |
| 25.0 | 0.488 | 8.4 | 783.9 | 784.5 | 100.1 | 94.9 |
| 27.5 | 0.558 | 8.5 | 849.6 | 781.6 | 92.0 | 95.1 |
| 30.0 | 0.439 | 8.5 | 933.4 | 845.8 | 90.6 | 95.5 |
| 32.5 | 0.472 | 8.5 | 900.8 | 839.7 | 93.2 | 95.6 |
| 35.0 | 0.513 | 8.6 | 870.2 | 851.6 | 97.9 | 95.5 |
| 37.5 | 0.450 | 8.6 | 869.3 | 896.3 | 103.1 | 96.0 |
| 40.0 | 0.509 | 8.6 | 868.8 | 790.9 | 91.0 | 95.9 |

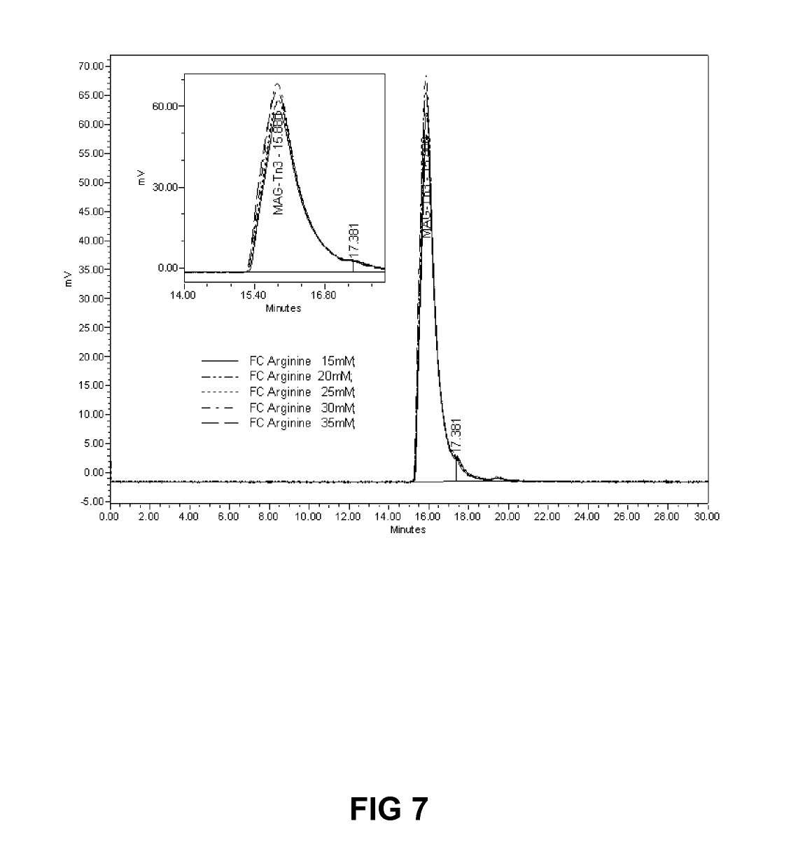

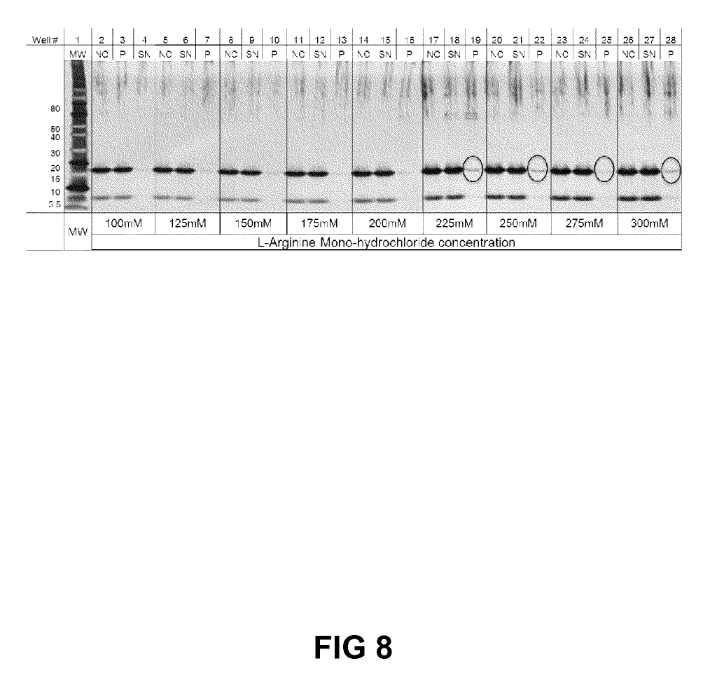

There is also no significant difference seen in % monomer by HPLC-SEC, all formulations are monomeric as illustrated in FIG. 7, with the major peak eluting at 15.9 minutes. 20 mM L-arginine was selected to screen the L-arginine monohydrochloride concentrations. Bands start to appear with more intensity in the pellet fractions at 225 mM L-arginine monohydrochloride, as seen in FIG. 8.

The turbidity results are very similar between the formulations, as seen in Table 8. Neither does the pH exhibit a lot of variation. A pH range of 7.8-8.2 is observed.

TABLE 8
Summary L-arginine monohydrochloride screening. A
summary of the results from L-arginine monohydrochloride
screening is provided.

| L-Arginine | | | | | | % |
|---|---|---|---|---|---|---|
| Mono- | | | MAG-Tn3 Concentration | | | Monomer |
| hydrochloride | | | by RP-HPLC (μg/mL) | | | by |
| Concentration | Turbidity | | | After | % Re- | HPLC- |
| (mM) | (NTU) | pH | Total | Filtration | covery | SEC |
| 100 | 0.434 | 8.2 | 870.8 | 816.2 | 93.7 | 94.0 |
| 125 | 0.386 | 8.2 | 868.0 | 709.4 | 81.7 | 95.3 |
| 150 | 0.394 | 8.0 | 776.9 | 864.4 | 111.3 | 95.2 |
| 175 | 0.429 | 8.0 | 800.2 | 829.8 | 103.7 | 95.3 |
| 200 | 0.367 | 8.0 | 914.9 | 669.1 | 73.1 | 95.0 |
| 225 | 0.410 | 7.9 | 877.5 | 752.4 | 85.7 | 94.9 |
| 250 | 0.374 | 7.8 | 876.7 | 699.0 | 79.7 | 95.2 |
| 275 | 0.339 | 7.8 | 792.3 | 777.1 | 98.1 | 95.2 |
| 300 | 0.439 | 7.8 | 866.5 | 839.9 | 96.9 | 94.9 |

The % MAG-Tn3 content recovery after filtration demonstrates more variability. L-arginine monohydrochloride concentrations of 125 mM and 200 mM-250 mM exhibit a significant loss in recovery of 30-15% indicating the presence of non-soluble aggregates. The 20% loss of recovery at 125 mM L-arginine monohydrochloride is potentially erroneous, since the recovery at 100 mM is 93.7%. The % recovery improves again at 275 mM and 300 mM L-arginine monohydrochloride, an explanation for this has yet to be understood.

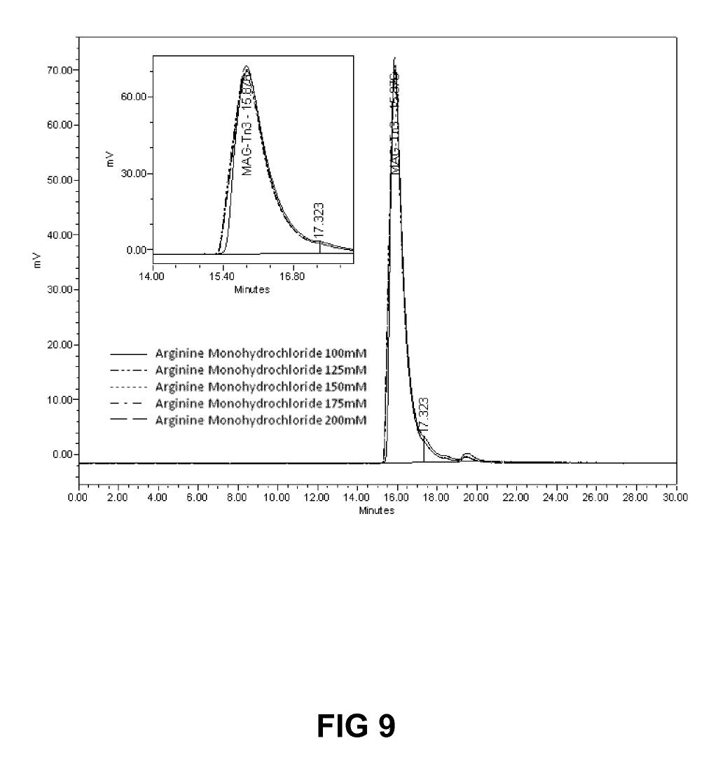

The remaining soluble portion of the L-arginine monohydrochloride formulations is monomeric. The aggregation profiles, as seen in FIG. 9, indicate a monomeric population.

### Discussion

L-Arginine concentrations from 17.5 mM to 40 mM proved to be stable. Both 12.5 and 15 mM L-Arginine exhibit a 10-20% loss in MAG-Tn3 recovery by RP-HPLC, suggesting the presence of insoluble aggregates. 20 mM L-arginine was selected as the buffer concentration since the stability of formulation between 15 and 17.5 mM is uncertain as these formulations have not been tested. 20 mM L-arginine allows for maneuverability, however; if the ±20% specifications are applied to 20 mM L-Arginine, the acceptable concentration range would become 16-24 mM. The stability of 16 mM should be assessed as 15 mM exhibits a loss of MAG-Tn3 content upon filtration of 10%.

The L-arginine monohydrochloride screening provided interesting results. The pH did not shift a lot in spite of the wide range of L-arginine monohydrochloride used, however; all of the pHs were less than 8.5 which is the pH to be avoided for its potential to deglycosylate the Tn sugars from MAG-Tn3. 150 mM L-arginine monohydrochloride is sufficient to lower the pH to 8.0 and maintain a stable formulation. There is no loss upon filtration, no band present in the pellet fraction by SDS-PAGE and the aggregation profile is monomeric for this formulation. The acceptance criteria for excipient concentrations when undergoing release testing is ±20%, this would put the range for L-arginine monohydrochloride at 120 mM-180 mM. The lower of which would appear to in a range of potential instability because the MAG-Tn3 recovery at 125 mM is 81.7%. This low value may be anomalous, since the recovery at 100 mM L-arginine monohydrochloride is 93.7%. Analyses should be performed to confirm the stability at 120 mM L-arginine monohydrochloride.

The final buffer composition for the 300 μg/dose MAG-Tn3 formulation with CpG7909 is 20 mM L-arginine and 150 mM L-arginine monohydrochloride.

### Example 4: Determination of a Maximum Dose for the MAG-Tn3 Antigen in an Arginine Buffer System

The targeted dose for the MAG-Tn3 antigen was set at 500 μg/dose. In the presence of the immunostimulant CpG7909, the MAG-Tn3 antigen co-precipitates instantaneously at this concentration. A soluble formulation is possible at a lower dose of 100 μg MAG-Tn3/dose in an arginine buffer system; however a higher dose would be preferable. In this report the experiments performed to determine the maximum MAG-Tn3 dose will be described.

The MAG-Tn3 vaccine formulation was initially targeted for a dose of 500 μg in a 500 μL injection volume containing 420 μg of the immunostimulant CpG7909. This formulation was not stable and resulted in a co-precipitation of the immunostimulant and the antigen. The co-precipitation was slightly mitigated with addition of histidine or arginine buffer systems.

In the previous experiment a soluble (non-precipitated) formulation was achieved by lowering the MAG-Tn3 antigen dose from 500 μg/dose to 100 μg/dose and using arginine as the buffer system. However a higher dose would be preferable.

The objective of this paper is to describe the experiments performed to determine the maximum dose of the glyco-peptide antigen, MAG-Tn3 in an arginine buffer system. The arginine buffer system used in this report is a mixture of L-arginine and L-arginine mono-hydrochloride. The L-arginine mono-hydrochloride is added in an attempt to lower the pH to a more favorable range for product stability and injectibility.

The initial experiment screened a large dose range of MAG-Tn3 from 200 μg/mL to 900 μg/mL. Once the upper limit was established, a smaller range of MAG-Tn3 doses was screened from 800 μg/mL to 900 μg/mL and finally a dose was selected.

### Experimental Procedures

Preparation of Formulations:

Using stock solutions of 500 mM L-Arginine and 1M L-Arginine mono-hydrochloride, formulations were made by adding 31.3 mM L-Arginine and 187.5 mM L-Arginine monohydrochloride to a 5% Sucrose solution in water for injection (Thermo-Fisher). CpG7909 (Agilent) was then added to the solution at a concentration of 1050 μg/mL. The solution was then magnetically stirred for 5 minutes at 150 rpm. MAG-Tn3 obtained from Lonza Braine was then added to the solutions at concentrations ranging from 250-1125 μg/mL. The solutions were then stirred magnetically for another 5 minutes at 150 rpm. The formulations were then diluted 1.25 times in a solution of 50 mM Na₂HPO₄/KH₂PO₄ 150 mM NaCl pH 6.1. All Formulations were then incubated for twenty-four hours at 4° C. before being analyzed. Chemicals were provided from Sigma-Aldrich.

Analyses:

RP-HPLC was performed to assess MAG-Tn3 content in the formulations before and after filtration with a 0.2 μm syringe filter. A Waters 2996 HPLC equipped with UV detection was used with a Poros R 1/10 column from Applied Biosciences and a 0-100% acetonitrile in 0.1% triflouroacetic acid gradient. Size-exclusion chromatography was performed using a Waters 2996 HPLC equipped with a fluorescence detector (Waters) to assess the aggregation profiles of the resulting formulations using a TSKgel G3000PWxl column (Tosoh Bioscience LLC) and a mobile phase of 200 mM NaCl. SDS-PAGE analysis was done using 4-12% Bis-Tris gels (Invitrogen) and MES running buffer (Invitrogen). The gels were stained using Invitrogen's SilverQuest. Centrifuged and non-centrifuged samples were run on gel to assess the presence of precipitate in the formulations. Centrifugation was performed for 15 minutes at 18 000 g. The supernatant was extracted after which the pellet was re-suspended in 1×LDS sample buffer (Invitrogen) and both fractions were run on gels. Turbidity (HACH), pH (Orion), and visual inspection analyses were also performed.

All formulations were found to be translucent and particle-free after the 24 hour incubation at 4° C. The pH and turbidity results were found to be similar between formulations; no significant difference was noted, as seen below in Table 9.

TABLE 9
Turbidity and pH. Turbidity and pH results from
200-900 ug/ml MAG-Tn3 dose screening.

| | MAG-Tn3 (μg/ml) | | | | | | | |
|---|---|---|---|---|---|---|---|---|
| | 200 | 300 | 400 | 500 | 600 | 700 | 800 | 900 |
| Turbidity | 0.389 | 0.433 | 0.421 | 0.528 | 0.347 | 0.402 | 0.430 | 0.343 |
| (NTU) | | | | | | | | |
| pH | 8.5 | 8.5 | 8.5 | 8.5 | 8.5 | 8.5 | 8.5 | 8.5 |

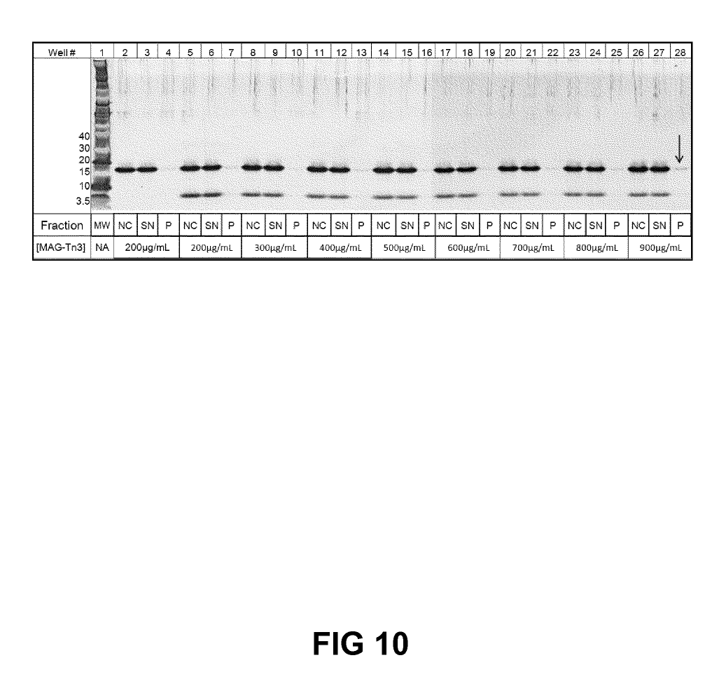

The SDS-PAGE results, visualized in FIG. 10, indicate a stable formulation up until a concentration of 900 μg/mL where there is the presence of a very slight band in the pellet fraction. This however is not detected as an insoluble aggregate as the % recovery of the filtered versus non-filtered sample is 100.0% by RP-HPLC, see Table 10.

TABLE 10
(A) MAG-Tn3 content. MAG-Tn3 antigen content determined by
RP-HPLC before and after filtration with a 0.2 μm syringe filter to
assess insoluble aggregate content and (B) recalculated by an alternate
method to correct ug/mL at higher concentrations.

| | MAG-Tn3 Concentration by RP-HPLC | | |
|---|---|---|---|
| | (μg/mL) | | |
| Targeted MAG-Tn3 | | After | % |
| Concentration (μg/mL) | Total | Filtration | Recovery |

(A)

| | | | |
|---|---|---|---|
| 200 | 214.1 | 206.5 | 96.5 |
| 300 | 217.8 | 195.7 | 89.8 |
| 400 | 211.9 | 204.1 | 96.3 |
| 500 | 211.8 | 208.8 | 98.6 |
| 600 | 219.6 | 208.3 | 94.9 |
| 700 | 218.0 | 198.3 | 91.0 |
| 800 | 211.2 | 202.1 | 95.7 |
| 900 | 223.3 | 223.3 | 100.0 |

(B)

| | | | |
|---|---|---|---|
| 200 | 214.1 | 206.5 | 96.5 |
| 300 | 327.5 | 294.2 | 89.8 |
| 400 | 423.7 | 408.1 | 96.3 |
| 500 | 528.1 | 520.7 | 98.6 |
| 600 | 655.6 | 621.9 | 94.9 |
| 700 | 757.7 | 689.2 | 91.0 |
| 800 | 844.7 | 808.4 | 95.7 |
| 900 | 1007.2 | 963.7 | 95.7 |

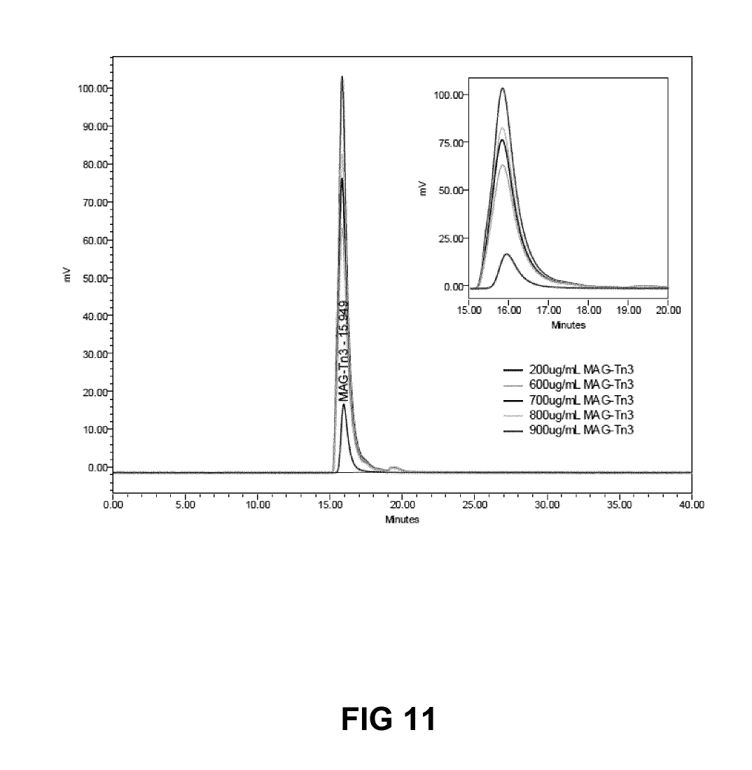

Size exclusion chromatography indicated no change in aggregation profile for all concentrations tested, as there was no shift in retention time observed, nor the appearance of peaks at shorter retention times indicating the presence of aggregates, as seen below in FIG. 11.

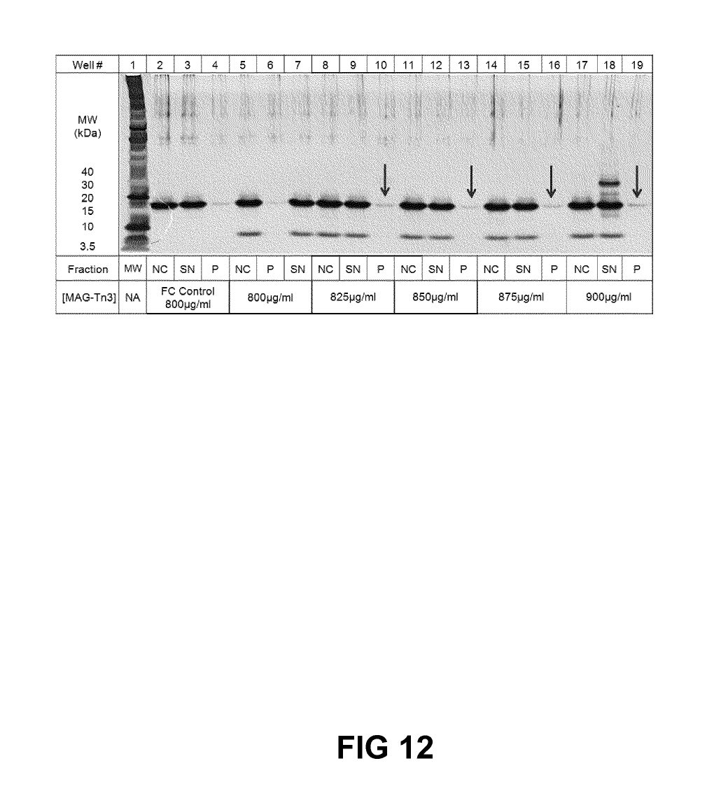

The narrower dose screening was performed between 800 and 900 μg/mL, the resulting gel can be seen below in FIG. 12. Slight bands can be seen in all pellet fractions including the control (purified MAG-Tn3 bulk); however, the bands are slightly more intense for the 825, 850, 875, 900 μg/mL MAG-Tn3 doses. The presence of the bands in the pellet fractions is not detected as insoluble aggregates since the % recovery of the filtered versus non-filtered samples are 100±10% (Table 11). Nor are any soluble aggregates observed as only the monomeric peak is observed by size exclusion chromatography.

TABLE 11
Dose range from 800-900 μg/mL. (A) A summary of results of dose
range from 800-900 μg/mL is provided and (B) recalculated by an
alternate method to correct ug/mL at higher concentrations.

| Targeted | | | | | | |
|---|---|---|---|---|---|---|
| MAG-Tn3 | | | MAG-Tn3 Concentration | | | % |
| Con- | | | (μg/mL) | | | Monomer |
| centration | Turbidity | | | After | % | by HPLC- |
| (μg/mL) | (NTU) | pH | Total | Filtration | Recovery | SEC |

(A)

| | | | | | | |
|---|---|---|---|---|---|---|
| 800 | 0.474 | 8.5 | 793.9 | 857.6 | 108.0 | 100.0 |
| 825 | 0.382 | 8.5 | 838.0 | 819.2 | 97.8 | 100.0 |
| 850 | 0.392 | 8.5 | 985.4 | 935.2 | 94.9 | 100.0 |
| 875 | 0.395 | 8.5 | 887.8 | 875.0 | 98.6 | 100.0 |
| 900 | 0.383 | 8.5 | 859.0 | 863.1 | 100.5 | 100.0 |

(B)

| | | | | | | |
|---|---|---|---|---|---|---|
| 800 | 0.474 | 8.5 | 793.9 | 857.6 | 108.0 | 95.7 |
| 825 | 0.382 | 8.5 | 870.9 | 851.3 | 97.8 | 100.0 |
| 850 | 0.392 | 8.5 | 1044.5 | 991.3 | 94.9 | 100.0 |
| 875 | 0.395 | 8.5 | 960.2 | 946.4 | 98.6 | 100.0 |
| 900 | 0.383 | 8.5 | 968.7 | 973.2 | 100.5 | 100.0 |

The MAG-Tn3 antigen appears soluble in the L-arginine-L-arginine-mono-hydrochloride buffer system up to a concentration of 900 μg/mL in the final reconstituted vaccine or 450 μg/dose. As the stability of MAG-Tn3 in the presence of CpG7909 is precarious as demonstrated by the multitude of attempts at solubilizing the two ingredients, a lower dose was selected.

During product stability and release testing the acceptance criteria for antigen content is set at 100±20%. The extreme 120% value must also be a soluble formulation, therefore if the upper limit of MAG-Tn3 solubility is 900 μg/mL, the centered MAG-Tn3 concentration would then be 750 μg/mL.

One must also take into consideration the dilution factor observed between the formulated bulk product and the reconstituted lyophilized cake. 500 μL of formulated bulk is lyophilized then reconstituted in a volume 625 μL, resulting in a dilution factor of 1.25. This also must be added to the calculation of the dose calculation. If 750 μg/ml is the maximum concentration that can be obtained, it would belong to the final bulk, the final container concentration would then be 600 μg/ml for a dose of 300 μg.

The maximum dose of MAG-Tn3 that is able to be formulated as a co-lyophilized product with the immunostimulant CpG7909 is 300 μg in an arginine buffer system. The arginine buffer system in these experiments was chosen based on pH; however, optimization of these components will be necessary.

### Example 5: Immunostimulant Dose Screening in the MAG-Tn3 Vaccine Formulation

The glyco-peptide MAG-Tn3 can be formulated in a soluble vaccine with the immunostimulant CpG7909 at a dose of 300 μg MAG-Tn3 and 380 μg CpG7909/mL in an L-arginine buffer system (for purposes of this discussion, the liquid volume of a dose is defined as 500 μL). A range of possible CpG concentrations within an acceptance criterion of 100±20% were investigated in this experiment to determine if MAG-Tn3 remains soluble in a range of CpG7909 doses.

The amount of CpG7909 was lowered from 420 μg/dose to 380 μg/dose based on a standard quantified by NMR. Utilizing the acceptance criteria of 100±20%, a CpG dose range of 300-460 μg/dose was investigated.

The objective of this report is to describe the experiment performed to make sure that the formulation remained stable. Formulations containing 180-420 μg CpG/dose were screen for stability.

### Experimental Procedures

Preparation of Formulations:

Using a stock solution of 250 mM L-Arginine (EMD) and 1875 mM L-Arginine monohydrochloride (Sigma Aldrich), formulations were made by adding 25 mM L-Arginine-187.5 mM L-Arginine monohydrochloride to a 5% Sucrose (EMD) solution in water for injection (Thermo-Fisher) and 0.1% Polysorbate 80 (NOF). CpG7909 (Agilent) was then added to the solution at a concentrations of 365-838 μg/mL. The solution was then magnetically stirred for 5 minutes at 150 rpm. MAG-Tn3 obtained from Lonza Braine was then added to the solutions at a concentration of 750 μg/mL. The solutions were then stirred magnetically for another 5 minutes at 150 rpm. The formulations were then diluted 1.25 times in a solution of 50 mM Na₂HPO₄/KH₂PO₄ 150 mM NaCl pH 6.1. All Formulations were then placed in the HPLC autosampler at 25° C. for injections at T0, 4 hours and 24 hours. These formulations are considered to be mock reconstituted final containers as they have not been through the lyophilization process.

Analyses:

Size-exclusion chromatography was performed using a Waters 2996 HPLC equipped with a fluorescence detector (Waters) to assess the aggregation profiles of the resulting formulations using a TSKgel Supermultipore PW-N guard and analytical column (Tosoh Bioscience LLC) with a 0.5 mL/in flow rate and a mobile phase of 20 mM L-arginine, 150 mM L-arginine-HCl, 0.08% polysorbate 80, 150 mM NaCl, 10 mM Na/K₂ phosphate buffer pH 6.1. Fluorescence detection was performed with an excitation wavelength of 270 nm and an emission wavelength of 318 nm.

### Results

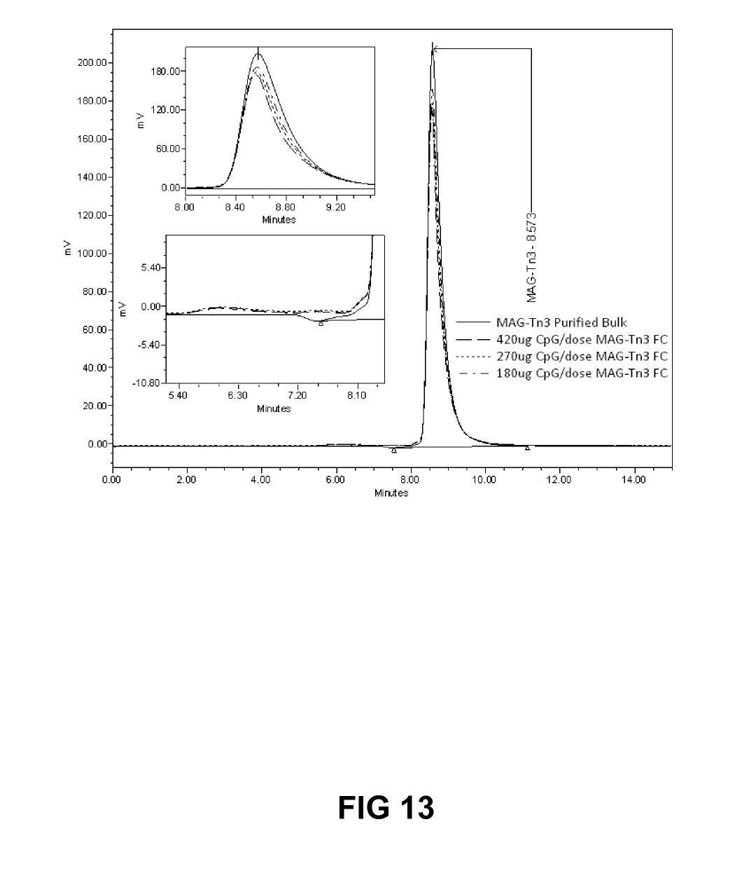

Size exclusion chromatography indicated no change in aggregation profile for all the CpG 7909 concentrations tested, as there was no shift in retention time observed for the major MAG-Tn3 monomeric peak at a retention time of 8.573 minutes. There is no change in the size of peaks at shorter retention times, indicating the slight presence of aggregates, when comparing the various CpG7909 concentrations as seen below in FIG. 13. A 350 and 230 ug CpG7909/dose were also tested and found to have a similar profile, results not shown.

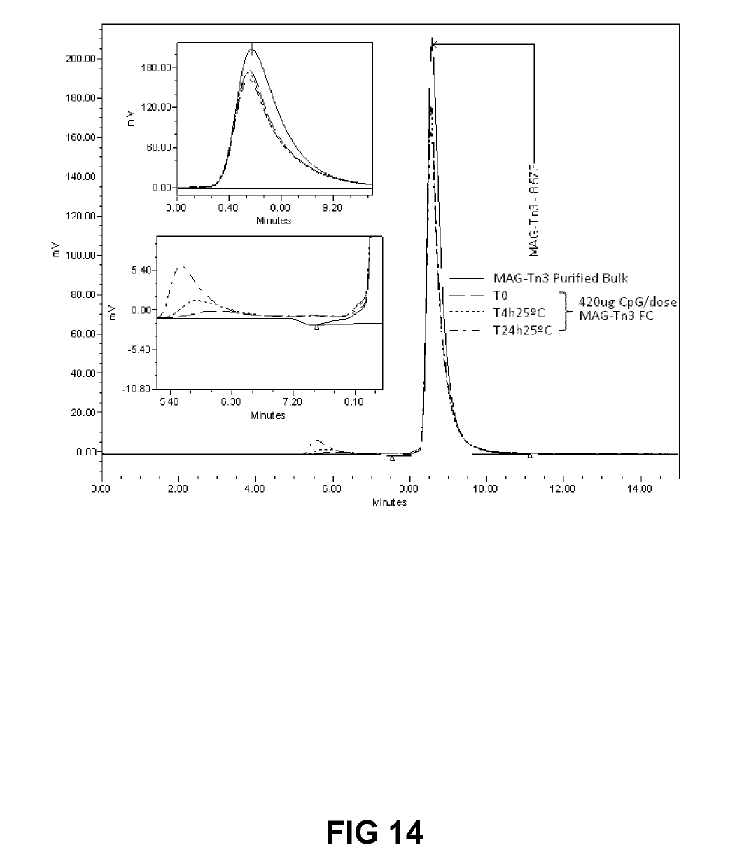

Incubation of the samples for 4 and 24 hours at 25° C. results in an evolution of the aggregation profile as seen in FIG. 14. The peak at 5.6 minutes, corresponding to aggregates, increases in peak area with respect to time spent at 25° C.

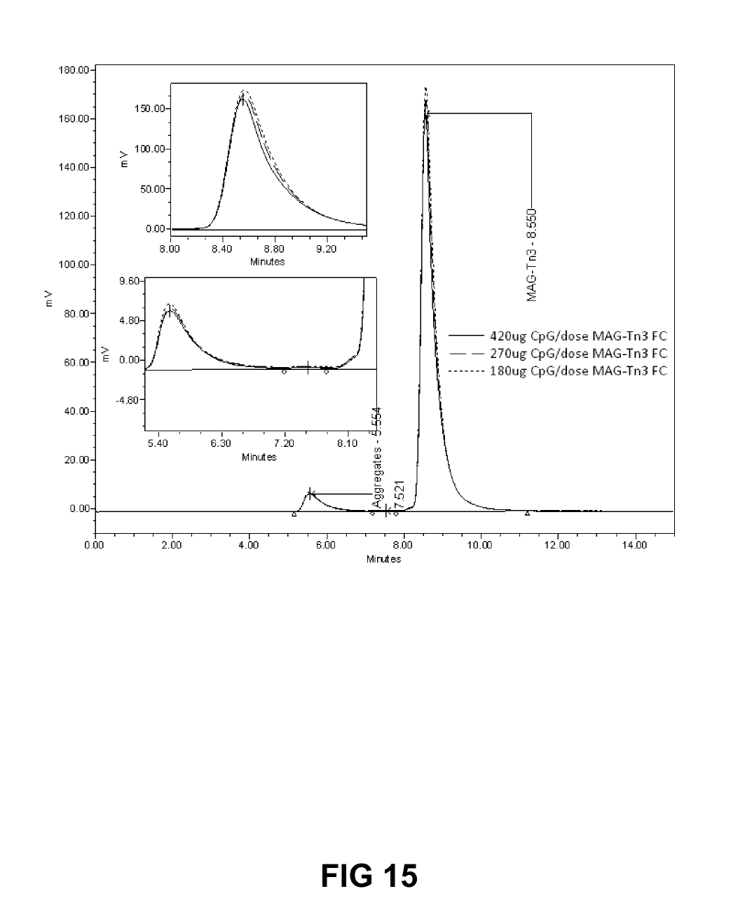

This phenomenon is observed at all CpG7909 doses tested as seen in FIG. 15. The 350 and 230 ug CpG7909/doses are not included in FIG. 15, but exhibit the same profile. The peak areas of both the aggregate peak and the monomeric peak (8.55 minutes) remain relatively constant, % CV of less than 10%, with increasing concentrations of CpG7909, as seen in Table 12.

TABLE 12
MAG-Tn3 monomer and aggregate peak area comparisons
at various CpG 7909 dose concentrations

| CpG | | | | | | |
|---|---|---|---|---|---|---|
| Concentration | Monomer | | | Aggregate | | |
| (μg/dose) | T0 | 4 h 25° C. | 24 h 25° C. | T0 | 4 h 25° C. | 24 h 25° C. |
| 420 | 4389470 | 4312017 | 4170588 | 52262 | 105071 | 251364 |
| 350 | 4511361 | 4430311 | 4322682 | 47834 | 106801 | 256292 |
| 270 | 4611544 | 4503391 | 4389977 | 46794 | 112739 | 250982 |
| 230 | 4700524 | 4569933 | 4449942 | 44161 | 115236 | 252104 |
| 180 | 4882667 | 4719013 | 4601093 | 44244 | 126648 | 267041 |
| Std Dev. | 187412 | 152334 | 158727 | 3319 | 8547 | 6761 |
| % CV | 4.06 | 3.38 | 3.62 | 7.05 | 7.54 | 2.65 |

The formulation containing 20 mM L-arginine and 150 mM L-arginine monohydrochloride creates a suitable matrix for the MAG-Tn3 antigen and the immunostimulant CpG7909 at concentrations ranging from 180 to 420 μg per dose. The CpG7909 bulk concentration is determined using an NMR quantified standard. Though an increase in aggregates is observed when incubated at 25° C., these aggregates are present at the same amounts regardless of CpG7909 concentration, suggesting the aggregates are due to antigen instability as opposed to an incompatibility with CpG7909 concentration. The upper limit of potential CpG7909 concentrations was not tested in this experiment; however the trend observed would indicate that a formulation containing 460 μg/mL CpG7909 would be stable.

### Example 6. pH Study

The calculated pH values for various mixtures of arginine and arginine hydrochloride were compared to the values determined with a pH meter (Table 13).

TABLE 13
Calculated vs. observed pH.

| | | | | | | | | |
|---|---|---|---|---|---|---|---|---|
| Arginine | 7 | 71 | 15 | 15 | 40 | 40 | 3 | 32 |
| con- | | | | | | | | |
| centration | | | | | | | | |
| (mM) | | | | | | | | |
| Arginine- | 220 | 100 | 465 | 45.4 | 124 | 1239 | 100 | 100 |
| HCl con- | | | | | | | | |
| centration | | | | | | | | |
| (mM) | | | | | | | | |
| Observed pH | 7.7 | 8.7 | 7.7 | 8.7 | 8.7 | 7.3 | 7.6 | 8.7 |
| Calculated | 7.5 | 8.5 | 7.5 | 8.5 | 8.5 | 7.5 | 7.5 | 8.5 |
| pH | | | | | | | | |
| pH | 0.2 | 0.2 | 0.2 | 0.2 | 0.2 | 0.2 | 0.1 | 0.2 |
| difference | | | | | | | | |

1-42. (canceled)

43. A substantially stable vaccine composition comprising:
(a) arginine;
(b) a counterion;
(c) a first immunogenic molecule comprising a Tn group, wherein the first immunogenic molecule is Mag-Tn3; and
(d) a second immunogenic molecule comprising an oligonucleotide, wherein the second immunogenic molecule is a CpG oligonucleotide;
said composition characterized in that when said composition comprises water (i) said first immunogenic molecule is substantially stable at a concentration of 48-720 μg/mL and said second immunogenic molecule is substantially stable at a concentration of 608-912 μg/mL; and (ii) the pH of the resulting solution is less than 8.5.

44. The composition of claim 43 further characterized in that when said composition comprises water, the arginine comprises the following species: 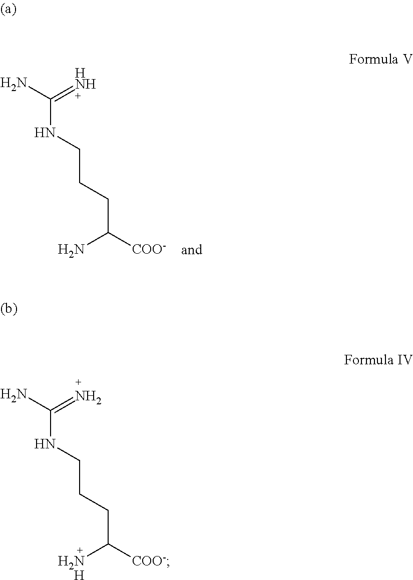
wherein the concentration of the species of (a) Formula V is at least 14 mM, and the molar ratio of the species of (a) Formula V to the species of (b) Formula IV is between 0.081 and 0.204.

45. The composition of claim 43 wherein the counterion is chloride and a portion of the arginine is present as the species of arginine monohydrochloride.

46. The composition of claim 43, wherein the MAG-Tn3 is present at a concentration of less than 900 μg/ml.

47. The composition of claim 43, comprising (i) between 60-900 μg/mL MAG-Tn3, inclusive; (ii) 950 μg/mL CpG 7909 (SEQ ID NO:4); (iii) 25 mM arginine; (iv) 187.5 mM arginine monohydrochloride; (v) 0.108% w/v Polysorbate 80; and (vi) 5% w/v sucrose.

48. The composition of 43, comprising (i) between 48-720 μg/mL MAG-Tn3, inclusive; (ii) 760 μg/mL CpG 7909 (SEQ ID NO:4); (iii) 20 mM arginine; (iv) 150 mM arginine monohydrochloride; (v) 0.0864% w/v Polysorbate 80; and (vi) 4% w/v sucrose.

49. The composition of claim 43 further comprising an adjuvant composition selected from the group consisting of:
(a) an adjuvant composition comprising one or more adjuvants, wherein at least one of said adjuvants is selected from the group consisting of MPL and QS21; and
(b) an adjuvant composition comprising liposomes and one or more adjuvants, wherein at least one of said adjuvants is selected from the group consisting of MPL and QS21.

50. The composition of claim 49, comprising (i) between 48-720 μg/mL MAG-Tn3, inclusive; (ii) 760 μg/mL CpG 7909 (SEQ ID NO:4); (iii) 20 mM arginine; (iv) 150 mM arginine monohydrochloride; (v) 0.0864% w/v Polysorbate 80; and (vi) 4% w/v sucrose; (vii) 150 mM NaCl; (viii) 8 mM KH₂PO₄ and 2 mM Na₂HPO₄; (ix) 50 μL/mL MPL; (x) 100 μg/mL liposomes; and (xi) 100 μg/mL QS21.

51. A process for making a substantially stable vaccine composition comprising the steps of combining the composition of 43 with a liquid comprising water.

52. A process for making a substantially stable vaccine composition comprising the steps of combining the composition of claim 43 with a liquid comprising water, wherein the liquid further comprises an adjuvant composition comprising one or more adjuvants, wherein at least one of said adjuvants is selected from the group consisting of MPL and QS21, and wherein the adjuvant composition further comprises liposomes.

53. A composition produced by the process of claim 52.

54. A method of treating a patient comprising the steps of administering a composition according to claim 53 to a human.

55. A method of inducing an immunogenic response comprising the steps of administering a composition according to claim 53 to a human.

56. A container comprising a composition according to claim 43.


SEQUENCE LISTING (6 sequences)

SEQ ID NO: 1 - DNA, 20 bp, Artificial Sequence
  synthesized
tccatgacgttcctgacgtt

SEQ ID NO: 2 - DNA, 18 bp, Artificial Sequence
  synthesized
tctcccagcgtgcgccat

SEQ ID NO: 3 - DNA, 30 bp, Artificial Sequence
  synthesized
accgatgacgtcgccggtgacggcaccacg

SEQ ID NO: 4 - DNA, 24 bp, Artificial Sequence
  synthesized
tcgtcgttttgtcgttttgtcgtt

SEQ ID NO: 5 - DNA, 20 bp, Artificial Sequence
  synthesized
tccatgacgttcctgatgct

SEQ ID NO: 6 - DNA, 22 bp, Artificial Sequence
  synthesized
tcgacgttttcggcgcgcgccg